(19) United States
(12) Patent Application Publication
(10) Pub. No.: US 2008/0138417 A1
(43) Pub. Date: Jun. 12, 2008

(54) Topical Composition And Method Of Forming

(76) Inventor: Charles Grigsby, Hamilton, IL (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/562,654

(22) Filed: Nov. 22, 2006

(51) Int. Cl. A61K 9/16; A61K 33/06; A61K 33/30; A61K 33/34; A61K 33/22; A61K 33/40

(52) U.S. Cl. 424/489; 424/682; 424/642; 424/657; 424/638; 424/613

(57) ABSTRACT

The present invention provides a topical composition for use in treating medical conditions such as fungal epithelial infections, carpal tunnel syndrome, tendonitis, and arthritis. The topical composition includes a mineral component including a source of calcium and a source of potassium, a vitamin component including a source of ascorbic acid, and an anti-inflammatory component including a source of lysine and a source of glutathione. The topical composition also includes an anti-oxidant component including a source of curcumin and an isoflavone component including a source of ipriflavone. The present invention also provides a method of forming the topical composition. The method includes combining the mineral component, the vitamin component, the anti-inflammatory component, the anti-oxidant component, and the isoflavone component.

### FIELD OF THE INVENTION

The present invention generally relates to a topical composition and a method of forming the topical composition. More specifically, the topical composition includes an isoflavone component including a source of ipriflavone.

### DESCRIPTION OF THE RELATED ART

Topical compositions used to treat medical conditions are well known in the art. Some medical conditions, such as epithelial and mucosal membrane conditions, may be caused by bacteria, fungi, parasites, and viruses. Other medical conditions, such as osteoporosis and joint inflammation may be caused, at least in part, by deficiencies in intake of nutrients such as vitamins, minerals, and amino acids. Topical compositions which effectively treat these various types of medical conditions typically include ingredients such as vitamins, minerals, and herbs. Yet, the efficacies of these compositions depend on the specific ingredients and their amounts in the compositions.

In the past, efforts have been made to develop compositions for specifically treating epithelial related conditions. One effort is disclosed in U.S. Pat. App. Pub. No. 2005/0277694 (the '694 application) and includes topical compositions and methods for treating epithelial related conditions. The topical compositions of the '694 publication include a variety of ingredients such as hormones (e.g., androgens, estrogens, or progesterones), vitamins, minerals, and additives. The hormones in the topical compositions are used to reduce inflammation. The vitamins are used as anti-oxidants. The minerals and additives are used as deodorants, protective, astringents, anti-fungal agents, and anti-dandruff agents. Yet, the topical compositions of the '694 publication do not include a source of isoflavones. Ipriflavone, a specific type of synthetic isoflavone, is an anti-oxidant, is thought to protect against certain types of cancers, is thought to interact with calcium to facilitate entry of nutrients into bone and bone marrow thereby improving overall health, and is contemplated that to effect many different physiological benefits. It is also contemplated that ipriflavone can be used to improve skin conditions. Because the compositions of the '694 publication do not include a source of ipriflavone, these compositions are lacking a useful ingredient that can be used to improve epithelial related conditions.

Although the aforementioned '694 publication does not include a source of isoflavones, other compositions, such as those used to treat hormone deficiencies, utilize sources of specific natural isoflavones. U.S. Pat. App. Pub. No. 2005/0226949 (the '949 publication) discloses a medical/nutritional composition for maintaining hormone balance. The medical/nutritional composition includes macronutrients such as proteins, carbohydrates, and lipids, and a source of micronutrients such as isoflavones and isoflavone synergists. Yet, the isoflavones utilized in the '949 publication do not include ipriflavone. Instead, the '949 publication focuses on and utilizes genistein, daidzein, and glycitein, which are naturally formed isoflavones. As is known in the art, genistein, daidzein, and glycitein are chemically distinct from ipriflavone, as ipriflavone includes an isopropoxy moiety at the 7 position of its phenyl chromanone (C₁₅H₁₂O₂) ring structure. Ipriflavone, due to this chemical distinctiveness as compared with genistein, daidzein, and glycitein, functions uniquely and provides unique physiological benefits. As such, like the compositions of the '694 publication, the compositions of the '949 publication are also missing a useful ingredient that is thought to effect many useful physiological benefits.

Accordingly, there remains an opportunity to form a topical composition that is useful for the treatment of a variety of medical conditions and that includes ipriflavone. There also remains an opportunity to utilize a method to form the topical composition.

### SUMMARY OF THE INVENTION AND ADVANTAGES

The present invention provides a topical composition for use in treating medical conditions. The topical composition includes a mineral component including a source of calcium and a source of potassium, a vitamin component including a source of ascorbic acid, and an anti-inflammatory component including a source of lysine and a source of glutathione. The topical composition also includes an anti-oxidant component including a source of curcumin and an isoflavone component including a source of ipriflavone. The present invention also provides a method of forming the topical composition. The method includes combining the mineral component, the vitamin component, the anti-inflammatory component, the anti-oxidant component, and the isoflavone component.

The calcium, ascorbic acid, potassium, lysine, glutathione, curcumin, and ipriflavone act via different mechanisms when used to treat medical conditions. It is hypothesized that at least two different mechanisms contribute to the effectiveness of the topical composition.

In a first mechanism, the calcium, the ascorbic acid, the potassium, the lysine, the glutathione, the curcumin, and the ipriflavone synergistically combine and interact to effectively treat medical conditions. Specifically, it is contemplated that the calcium interacts with the ipriflavone to increase absorption of the calcium into bone marrow. It is also contemplated that the potassium absorbs into blood. Further, it is contemplated that the ascorbic acid, lysine, glutathione, and curcumin, act as anti-oxidants and anti-inflammatory agents. Combined, it is contemplated that each act together to effectively treat a variety of medical conditions.

In a second mechanism, a solubility of each of the sources of the calcium, the ascorbic acid, the potassium, the lysine, the glutathione, the curcumin, and the ipriflavone, under physiological conditions, increases a likelihood that each of the calcium, the ascorbic acid, the potassium, the lysine, the glutathione, the curcumin, and the ipriflavone will be absorbed quickly and effectively into blood and/or bone marrow. It is contemplated that quick and effective absorption increases a speed and efficacy of the topical composition in treating the variety of medical conditions.

Specifically when the topical composition is used to control and eliminate fungal epithelial infections, it is contemplated that a third mechanism may be involved. In the third mechanism, the topical composition reduces an oxygen supply to a fungus thereby assisting in the control and elimination of the fungal epithelial infections. As such, the third mechanism is complementary to the first and second mechanisms by combining both mechanical and chemical effects.

### DETAILED DESCRIPTION OF A PREFERRED EMBODIMENT

The present invention provides a topical composition (composition) for use in treating medical conditions. The composition may be used to effectively control and eliminate epithelial infections and inflammations. Non-limiting examples of one type of epithelial infection are fungal epithelial infections which include, but are not limited to, dermatophyte infections, tinea corporis, tinea pedis, tinea unguium, tinea capitis, tinea cruris, tinea versicolor, tinea barbae, athlete's foot, and jock itch. Non-limiting examples of other types of epithelial infections and inflammations include atopic dermatitis, medicamentosa, contact dermatitis, seborrheic, nummular dermatitis, exfoliative stasis, acne vulgaris, nodulocystic acne, acne fulminans, steroid acne, acne keloidalis nuchae, chloracne, pyoderma faciale, and cysts, scalp folliculitis, spa pool folliculitis, oil folliculitis, pityrosporum folliculitis, gram negative folliculitis, pseudofolliculitis barbae, chilblains, miliaria, tinea rosacea, steroid rosacea perioral dermatitis, eczema, psoriasis, staphylococcal diseases such as staphylococcal scalded skin syndrome, erysipelas, folliculitis, furuncles, carbuncles, paronychial infections, erythrasma, crodermatitis enteropathica, Sweet's disease, lichen amyloidosis, macular amyloidosis, acute generalized and chronic generalized hives and physical hives, erythema annulare centrifugum, annular erythema such as erythema perstans, erythema gyratum perstans, erythema gyratum repens and erythema figuratum pertans, uveitis, erythema nodosum, biotin response, dermatoses, pyoderma gangrenosum, erythema multiforme, aphthous ulcers, granulomatous cheilitis, dermitis herpetiformis, dermatomyositis, eosinophilic fascitis, sea bather's eruption, seaweed dermatitis, swimmers itch, scombroid fish poisoning, scabies, popular urticaria, cutaneous larva migrans, candida albicans, oral candida, candidal paronychia, scabies, pediculosis capitis, pediculosis corporis, pediculosis pubis, herpes simplex, herpes zoster, chicken pox, measles, rubella, bullous disease, burns, cysts, hidradenitis suppurativa, and cellulitis. It is contemplated that the composition of the instant invention may be effective in treating any or all of the aforementioned conditions.

The composition may be used to treat carpal tunnel syndrome and/or arthritis, of which there are over 100 varieties including, but not limited to, osteoarthritis and rheumatoid arthritis. The composition may also be used to treat tendonitis. However, the composition is not limited to such applications. It is contemplated that the composition, depending on what is included, may be used to treat bacterial and viral epithelial infections, may be used in the treatment of bone diseases and ailments, may be used in anti-oxidant and anti-inflammatory treatments, and may be used as a nutrient to treat vitamin and mineral deficiencies.

The composition may be applied to any animal. Specifically, the composition may be used in a method of treating a fungal epithelial infection that includes the step of applying the composition to the animal. Preferably, the animal includes, but is not limited to, mammals such as dogs, cats, horses, and humans, and other animals such as fish, reptiles, birds, and amphibians. Most preferably, the composition is applied to humans. Additionally, the composition may be applied to any substrate that the animal contacts such as a cage, stall, bed, blanket, or item of clothing such as a shoe. The composition may also be applied to any part of the animal. As the composition is a topical composition, the composition may be applied to any skin and/or mucous membrane surface. Preferably, the composition is applied to a skin surface.

The composition includes a mineral component including a source of calcium and a source of potassium, a vitamin component including a source of ascorbic acid, and an anti-inflammatory component including a source of lysine and a source of glutathione. The topical composition also includes an anti-oxidant component including a source of curcumin and an isoflavone component including a source of ipriflavone. Each will be described in greater detail below.

The mineral component includes the source of calcium and the source of potassium and may include a separate source of an additional mineral selected from the group of zinc, boron, manganese, magnesium, copper, selenium, and combinations thereof.

The mineral component including the source of the calcium and potassium, and optionally the separate source of the additional mineral including the zinc, boron, manganese, magnesium, copper, and/or selenium, may be present elementally, as pharmaceutically acceptable salts, as complex ions in solution, in complexes with various ligands, and/or as oxides such as hydroxides and peroxides. As is known in the art, the terminology “pharmaceutically acceptable salt” includes salts prepared from pharmaceutically acceptable non-toxic acids and bases including inorganic and organic acids. Non-limiting examples of such inorganic acids include hydrochloric acid, hydrobromic acid, hydriodic acid, sulfuric acid, phosphoric acid, and combinations thereof. Non-limiting examples of organic acids include aliphatic acids, aromatic acids, carboxylic and sulfonic acids such as formic acid, acetic acid, propionic acid, succinic acid, glycolic acid, glucuronic acid, maleic acid, furoic acid, glutamic acid, benzoic acid, anthranilic acid, salicylic acid, phenylacetic acid, mandelic acid, embonic acid, methanesulfonic acid, ethanesulfonic acid, pantothenic acid, benzenesulfonic acid, stearic acid, sulfanilic acid, algenic acid, galacturonic acid, and combinations thereof.

In one embodiment, the source of the calcium and potassium, and also the optional separate source of the additional mineral including the zinc, boron, manganese, magnesium, copper, and/or selenium include a salt of cholic acid such that the mineral is cholated. For descriptive purposes only, a chemical structure of a cholated mineral is shown below wherein M can be calcium, potassium, zinc, boron, manganese, magnesium, copper, and/or selenium and n is an integer that is the same as a positive valence charge of M:

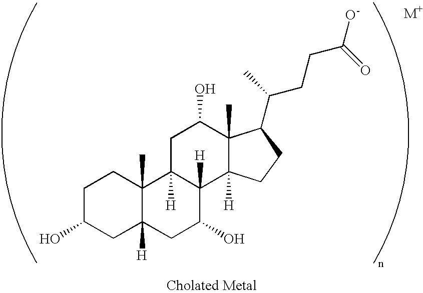

Referring to the source of calcium, this source may include, but is not limited to, elemental calcium (Ca), calcium salts such as calcium citrate (C₁₂H₁₀Ca₃O₁₄), calcium lactate (C₆H₁₀CaO₆), and calcium carbonate (CaCO₃), oxides such as hydroxides and peroxides, as microcrystalline calcium hydroxyapatite (Ca₁₀(PO₄)₆(OH)₂), as complex ions in solution, in complexes with various ligands, and combinations thereof. If the source of calcium includes the microcrystalline calcium hydroxyapatite, the hydroxyapatite may include protein including collagen, elemental calcium, and phosphorus. The collagen may be any type including Type I and Type V collagen.

The source of calcium may have any solubility. Preferably, the source of calcium has a solubility of at least 10 percent by weight, measured at physiological conditions, as known in the art. In one embodiment, the source of calcium includes calcium lactate, and is commercially available from Jungbauer Inc. of Newton Centre, Mass., under the trade names of Purecal T, Purecal O, and Purecal U. In another embodiment, the source of calcium includes cow's milk available from Glambia Foods Inc., of Monroe, Wis. In yet another embodiment, the source of calcium is preferably present in the composition in an amount of from 0.2 to 0.4, more preferably of from 0.3 to 0.4, and most preferably of from 0.35 to 0.4, parts by weight per 100 parts by weight of the composition. The source of calcium may be present in any effective amount and may be present in an amount that is as high as practical, as determined by one skilled in the art, depending on use of the composition and medical condition being treated.

As is known in the art, calcium, whose primary physiological form is hydroxyapatite, may be absorbed into blood, bone, and bone marrow. As is also known in the art, calcium may be physiologically absorbed by both active and passive transfer, may interact with vitamin D, and is also involved in effective muscle contraction, nerve impulse transmission, blood clotting, regulation of hormone secretion, and enzyme function. Without intending to be limited by any particular theory, it is contemplated that absorption of calcium increases bone density and increases therapeutic effectiveness of the composition when used to treat arthritis, especially rheumatoid arthritis, and fungal epithelial infections.

Referring now to the source of the potassium, this source of potassium may include potassium in any form including, but not limited to, elemental potassium (K), potassium salts such as potassium hydroxide (KOH), and combinations thereof. The source of potassium is commercially available from Spectrum Chemical Mfg Corp. of Gardena, Calif. and is preferably included in an amount such that the pH of the composition is above 7 and most preferably approximately 7.42. In one embodiment, the pH of the composition is at least 10. The source of potassium is preferably present in the composition in an amount of from 0.1 to 0.5, more preferably of from 0.1 to 0.2, and most preferably of from 0.15 to 0.17, parts by weight per 100 parts by weight of the composition. However, one skilled in the art may include the source of potassium in the composition in any amount, depending on use of the composition and medical condition being treated.

Potassium may absorb into blood and bone marrow and aid in regulation of nerve function. As is known in the art, potassium also functions in many other beneficial ways as an electrolyte, in blood pressure regulation, and in heart function. Potassium salts may neutralize acids and enter bloodstreams, thereby synergistically working with sodium to maintain and/or restore membrane potentials and to assist in metabolic processes. Potassium further regulates transfer of nutrients into cells, facilitates creation of energy in the form of ATP, and regulates cellular water balance. Still further, it is contemplated that potassium also aids rheumatic and arthritic conditions by promoting the release of acids from joints thereby easing stiffness. Potassium is also involved in elimination of wastes, acts as a natural pain desensitizer, mitigates convulsions, headaches and migraines, promotes healing of abrasions, and generally contributes to a sense of well being. It is contemplated that each of these aforementioned physiological processes may be improved through use of the composition.

Referring now to the separate source of the additional mineral that may be included in the composition, this additional mineral may include an elemental mineral or a mineral compound. The additional mineral may include zinc such as elemental zinc powder (Zn) and/or zinc compounds such as zinc sulfate (ZnSO₄), cholated zinc, zinc oxide (ZnO), zinc ascorbate (C₁₂H₁₄ZnO₁₂.2H₂O), zinc glycinate (C₄H₈N₂O₄Zn), zinc aspartate (C₄H₅NO₄Zn), zinc arginate (C₁₂H₂₆N₈O₄Zn), zinc citrate (C₁₂H₁₀O₁₄Zn₃), zinc gluconate (C₁₂H₂₂O₁₄Zn), zince picolinate (C₁₂H₈N₂O₄Zn), and combinations thereof. The additional mineral may include cholated zinc which is commercially available from Spectrum Chemical Mfg Corp. of Gardena, Calif. In one embodiment, the source of the additional mineral including the zinc is preferably present in an amount of from 0.05 to 0.2, more preferably of from 0.05 to 0.15, and most preferably of from 0.10 to 0.11, parts by weight per 100 parts by weight of the composition. The source of the additional mineral including the zinc may be present in any effective amount, as determined by one skilled in the art, depending on use of the composition and medical condition being treated.

As is known in the art, zinc may function as an astringent, an antiseptic and a skin protecting agent (e.g., a sun block) and may also act as an anti-inflammatory. As an anti-inflammatory, zinc may be used to relieve symptoms of arthritis. Zinc is also physiologically utilized in immune system function (e.g., with T-lymphocyte activity), in wound healing, in growth and development, in sexual maturation and reproduction, in dark vision adaptation, in olfactory and gustatory activity, in calcium absorption into bone marrow, and in DNA synthesis and gene regulation. Further, zinc is also known to be present in many physiologically important metalloenzymes such as carbonic anhydrase, carboxypeptidases A and B, alcohol dehydrogenase, glutamic dehydrogenase, D-glyceraldehyde-3-phosphate dehydrogenase, lactic dehydrogenase, malic dehydrogenase, alkaline phosphatase, and aldolase. Without zinc, synthesis of nucleic acids and proteins may be impaired. It is also contemplated that zinc may be involved in secretion and function of insulin and that use of the composition including zinc may improve any or all of the aforementioned physiological processes.

The additional mineral may alternatively include boron such as elemental boron (e.g., boron powder). The boron is commercially available from Spectrum Chemical Mfg Corp. of Gardena, Calif. In one embodiment, the source of the additional mineral including the boron is preferably present in an amount of less than 0.001 parts by weight per 100 parts by weight of the composition. It is contemplated that the source of the additional mineral including the boron may be present in any effective amount, as determined by one skilled in the art, depending on use of the composition and medical condition being treated.

Boron may be used to relieve symptoms of osteoarthritis, osteoporosis, and psoriasis and may be used in treatment of epithelial infections and treatment of excessive blood clotting. Boron may physiologically act to regulate absorption of calcium, improves bone density, and slows progression of osteoporosis. Specifically, boron may also contribute to cell-membrane function, trans-membrane signaling, trans-membrane movement of ions such as sodium and potassium, transport of extracellular calcium, and release of intracellular calcium in platelets. Further, boron may also act as a metabolic regulator in immune systems and may form complexes with carbohydrates, nucleotides, and vitamins. Use of the composition including the boron may improve any or all of the aforementioned physiological processes.

The additional mineral may also include manganese including, but is not limited to, elemental manganese (Mn), manganese sulfate (MnSO₄), manganese glycinate (C₄H₈MnN₂O₄), manganese gluconate C₁₂H₂₂MnO₁₄), manganese arginate (C₁₂H₂₆MnN₈O₄), manganese aspartate (C₄H₅MnNO₄), manganese chelated with ethylenediaminetetraacetic acid, and combinations thereof. The manganese is commercially available from Spectrum Chemical Mfg Corp. of Gardena, Calif. In one embodiment, the source of the additional mineral including the manganese is preferably present in an amount of from 0.01 to 0.05, more preferably of from 0.01 to 0.03, and most preferably of from 0.02 to 0.03, parts by weight per 100 parts by weight of the composition. However, the source of the additional mineral including the manganese may be present in any effective amount, as determined by one skilled in the art, depending on use of the composition and medical condition being treated.

As is known in the art, manganese is an essential trace mineral in nutrition and is utilized in growth processes, in formation of bone and cartilage, in absorption of calcium into bone marrow, in reproductive development, in sensory function, and in insulin metabolism. As is also known in the art, manganese is a metal cofactor in glycosyltransferases, which function in the synthesis of glycoproteins and glycosaminoglycans. Manganese also functions in metalloenzymes such as manganese superoxide dismutase, which is an anti-oxidant. Further, manganese may exhibit anti-osteoporotic and anti-arthritic properties which may be realized upon use of the instant composition.

The additional mineral may also include magnesium, as first introduced above. The magnesium may include, but is not limited to, elemental magnesium (Mg), magnesium oxide (MgO), magnesium glycinate (C₄H₈MgN₂O₄), magnesium citrate (C₁₂H₁₀Mg₃O₁₄), magnesium aspartate (C₄H₅MgNO₄), magnesium malate (C₄H₄MgO₅), dihydrogen magnesium ethylenediaminetetraacetate (C₁₀H₁₄N₂O₈Mg), and combinations thereof. In one embodiment, the source of the additional mineral including the magnesium is preferably present in an amount of from 0.01 to 0.15, more preferably of from 0.02 to 0.1, and most preferably of from 0.06 to 0.1, parts by weight per 100 parts by weight of the composition. In one embodiment, the source of the additional mineral includes magnesium sulfate (MgSO₄). The magnesium sulfate may be present in the same amounts as the aforementioned source of magnesium. It is to be appreciated that that the source of the additional mineral including the magnesium may be present in any effective amount, as determined by one skilled in the art, depending on use of the composition and medical condition being treated.

Magnesium is present in intracellular fluids and is involved in numerous biochemical reactions and processes such as production of cellular energy, synthesis of nucleic acids and proteins, maintenance of cell membranes, muscle contraction, and nerve function. Magnesium is may also function both antagonistically and complementarily with calcium. For example, in synthesis of nucleic acids and protein, calcium and magnesium are antagonistic. Conversely, in production of adenosine triphosphate, magnesium and calcium work complementarily. Additionally, magnesium may have anti-osteoporotic, anti-arrhythmic, anti-hypertensive, glucose-regulatory, myocardial protective, anti-migraine, and bronchodilatory activity and any or all of these aforementioned physiological processes may be improved through use of the composition including magnesium.

The additional mineral may also include copper which may be found in copper sulfate (CuSO₄), copper oxide (CuO), copper glycinate (C₄H₈CuN₂O₄), copper gluconate (C₁₂H₂₂CuO₁₄), copper lysinate (C₁₂H₂₆CuN₄O₄), copper tyrosinate (C₁₈H₂₀CuN₂O₆, and combinations thereof. In one embodiment, the source of the additional mineral including the copper is preferably present in an amount of from 0.01 to 0.05, more preferably of from 0.01 to 0.04, and most preferably of from 0.02 to 0.03, parts by weight per 100 parts by weight of the composition. In another embodiment, the source of the additional mineral including the copper includes copper sulfate and is present in 0.001 parts by weight per 100 parts by weight of the composition. In yet another embodiment, the source of the additional mineral including the copper includes copper gluconate and is present in 0.022 parts by weight per 100 parts by weight of the composition. For descriptive purposes only, a structure of copper gluconate is shown below:

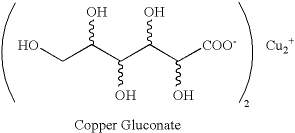

### It is contemplated that the source of the additional mineral including the copper may be present in any effective amount, as determined by one skilled in the art, depending on use of the composition and medical condition being treated.

Copper is an important trace element in diets and is known to function in many copper based metalloenzymes such as tyrosinase, ascorbic acid oxidase, lactase, cytochrome oxidase, uricase, monoamine oxidase, 6-aminolevulinic acid hydrydase, and dopamine-β-hydroxylase. It is also contemplated that copper functions in absorption and utilization of iron, electron transport, connective tissue metabolism, phospholipid formation, purine metabolism, and development of the nervous system. Copper may also exhibit anti-oxidant activity, have anti-fungal properties, and be used to protect cells and DNA from oxidative damage. If the additional mineral includes the copper, any or all of the aforementioned physiological processes may be improved.

Referring now to selenium, the source of the additional mineral including the selenium may be present in an amount of from 0.1 to 0.2 and is more preferably present in an amount of from 0.1 to 0.12, parts by weight per 100 parts by weight of the composition. It is contemplated that the source of the additional mineral including the selenium may be present in any effective amount, as determined by one skilled in the art, depending on use of the composition and medical condition being treated.

In one embodiment, the selenium is present in the composition to add color to the composition. However, selenium is known to have anti-oxidant, immunomodulatory, anti-carcinogenic and anti-atherogenic properties and may contribute to physiological defense against the toxicity of reactive oxygen species, regulation of thyroid hormone metabolism, and regulation of cellular redox reactions. Selenium is also a component of enzymes which protect cell membranes from free radical generation and oxidation. Additionally, selenium may contribute to tissue elasticity, prevention of dandruff, and to reduction of risks of breast, colon, lung and prostate cancers and each of the aforementioned physiological processes may be improved through use of the composition.

Referring back to the composition, the composition also includes the vitamin component including the source of ascorbic acid. The source of ascorbic acid may include, but is not limited to, L and/or D enantiomers of ascorbic acid and combinations thereof. Preferably, the source of ascorbic acid includes the L-enantiomer of ascorbic acid, commonly known as vitamin C. For descriptive purposes only, the L-enantiomer of ascorbic acid is shown below:

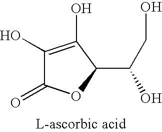

The vitamin component may also include additional compounds related to the L and D enantiomers of ascorbic acid including, but not limited to, ascorbic-2-sulfate (C₆H₈O₉), ascorbate-2-polyphosphate (C₆H₉O₉P), niacinamide ascorbate (C₁₂H₁₄N₂O₇), xyloascorbic acid (C₆H₈O₆), ascorbyl palmitate (C₂₂H₃₈O₇), mineral ascorbates such as sodium ascorbate (C₆H₇NaO₆), calcium ascorbate (Ca(C₆H₇O₆)₂), potassium ascorbate (C₆H₇KO₆), magnesium ascorbate (Mg(C₆H₇O₆)₂), zinc ascorbate (Zn(C₆H₇O₆)₂), molybdenum ascorbate (Mo(C₆H₇O₆)₆), chromium ascorbate (Cr(C₆H₇O₆)₆), manganese ascorbate (Mn(C₆H₇O₆)₂), and combinations thereof. Preferably, the source of ascorbic acid includes vitamin C and is commercially available from Spectrum Chemical Mfg Corp. of Gardena, Calif. In one embodiment, the source of ascorbic acid is preferably present in the composition in an amount of from 0.2 to 0.4, more preferably of from 0.2 to 0.3, and most preferably of from 0.2 to 0.25, parts by weight per 100 parts by weight of the composition. It is contemplated that the source of ascorbic acid may be present in any effective amount, as determined by one skilled in the art, depending on use of the composition and medical condition being treated.

It is contemplated that vitamin C has anti-oxidant, anti-atherogenic, anti-carcinogenic, anti-hypertensive, anti-viral, anti-histaminic, immunomodulatory, opthalmoprotective and airway-protective properties, and may be absorbed into blood, bone, and bone marrow. Vitamin C may also function in detoxification of heavy metals and may minimize cellular oxidation, aging, tissue and organ damage, and heart disease. As is known in the art, vitamin C is used for the formation of intercellular collagen, is vital to maintenance of tooth structures, matrix of bone, and walls of capillaries, is useful in healing of bone fractures and wounds, is an effective water-soluble antioxidant in human plasma, and is used for the proper immune system functioning and production of white blood cells, T-cells, and macrophages. As is also known in the art, vitamin C is a cofactor for prolyl and lysyl hydroxylase, enzymes used in the formation of collagen. Typically, collagen synthesized in the absence of vitamin C is not properly formed resulting in blood-vessel fragility. Vitamin C is also involved in the biosynthesis of other connective-tissue components, including elastin, fibronectin, proteoglycans, bone matrix and elastin-associated fibrillin and may contribute to collagen gene expression and cellular procollagen secretion. Further, vitamin C may modulate iron absorption, transport and storage. It is contemplated that, through application of the instant composition, each of the aforementioned physiological processes may be improved.

In addition to the source of ascorbic acid, the vitamin component may also include a source of an additional vitamin selected from the group of vitamin E, vitamin D, vitamin K, vitamin B, and combinations thereof. In a preferred embodiment, the composition includes a source of vitamin E, a source of vitamin D, a source of vitamin K, and a source of vitamin B, in addition to the source of ascorbic acid.

As is known in the art, vitamin E is a generic term and includes a group of chemically-similar compounds sharing tocopherol and/or tocotrienol structures. As such, the source of the vitamin E in the present composition may include compounds including one or both of these structures and any or all of the various isomers. For descriptive purposes only, chemical structures of α-tocopherol and tocotrienol are shown below:

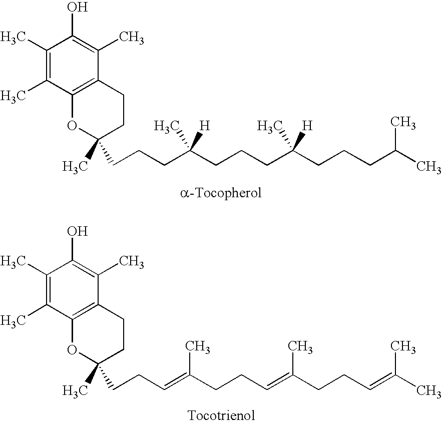

As illustrated above, both the α-tocopherol and the tocotrienol structures are similar except that the tocotrienol structure has double bonds on isoprenoid units. Specifically, in α-tocopherol, there are three chiral centers, at positions 2′, 4′, and 8′, such that there are eight stereoisomers.

There are also many other derivatives of these aforementioned structures which may be included in the source of the vitamin E. These derivatives result from different methyl groups disposed on the aromatic rings of both the tocotrienol and tocopherol structures at positions 5, 6, 7, and 8. If the methyl groups are disposed at positions 5, 7, and 8, the tocotrienol is α-tocotrienol. If the methyl groups are disposed at positions 5 and 8, the tocopherol is β-tocopherol and the tocotrienol is β-tocotrienol. If the methyl groups are disposed at positions 7 and 8, the tocopherol is τ-tocopherol and the tocotrienol is τ-tocotrienol. If a methyl group is disposed at position 8, the tocopherol is δ-tocopherol and the tocotrienol is δ-tocotrienol.

It is also contemplated that the source of the vitamin E may include synthetic derivatives of vitamin E including racemic-α-tocopherol, also known as 2,5,7,8-tetramethyl-2RS-(4⁹ RS, 8⁹RS, 12⁹ trimethyltridecyl)-6-chromanol, four 2R-stereoisomers including RRR-α-tocopherol, RSR-α-tocopherol, RRS-α-tocopherol and RSS-α-tocopherol, and four 2S-stereoisomers including SRR-α-tocopherol, SSR-α-tocopherol, SRS-α-tocopherol and SSS-α-tocopherol, and combinations thereof. Further, the vitamin component may include acetate and succinates of the tocopherols and/or tocotrienols. Still further, the source of the vitamin E may include vitamin E present in a cholated form. Most preferably, the source of vitamin E includes vitamin E that is made into a salt or ester of cholic acid.

As known by those of skill in the art, vitamin E is an anti-oxidant, contributes to a healthy circulatory system, aids calcium entry into bone marrow, aids in proper blood clotting, improves wound healing, and may have anti-atherogenic, anti-thrombotic, anti-coagulant, neuroprotective, anti-proliferative, immunomodulatory, cell membrane-stabilizing and anti-viral properties. Vitamin E can also alleviate fatigue by increasing blood oxygen supplies, can facilitate cell nourishment, can strengthen capillary walls, can prevent and dissolve blood clots, and can retard muscular dystrophy. Each of the aforementioned physiological processes may be improved through use of the composition.

In one embodiment, the source of vitamin E is preferably present in the composition in an amount of approximately 5.6 IU per 100 grams of the composition and is commercially available from Spectrum Chemical Mfg Corp. of Gardena, Calif. However, the source of vitamin E may be present in any effective amount, as determined by one skilled in the art, depending on use of the composition and medical condition being treated.

Referring now to the source of the vitamin D, in one embodiment, the source is preferably present in the composition in an amount of approximately 111 IU per 100 grams of the composition and is commercially available from Spectrum Chemical Mfg Corp. of Gardena, Calif. It is to be appreciated that the source of vitamin D may be present in any effective amount, as determined by one skilled in the art, depending on use of the composition and medical condition being treated. As is well known in the art, vitamin D is a steroid hormone that is commonly known as a fat soluble vitamin. Vitamin D exists in several forms including vitamin D₁, vitamin D₂, vitamin D₃, vitamin D₄, and vitamin D₅. Vitamin D₁ is a molecular compound of ergocalciferol and lumisterol in a 1:1 ratio. Vitamin D₂ is known as ergocalciferol or calciferol. Vitamin D₃ is known as cholecalciferol, calcidiol, and calcitriol. Vitamin D₄ is 22,23-dihydroergocalciferol. Vitamin D₅ is sitocalciferol, made from 7-dehydrositosterol. The source of vitamin D in the instant invention may include any or all of the forms of vitamin D known in the art. In one embodiment, the source of the vitamin D includes vitamin D₃. For descriptive purposes only, chemical structures of vitamins D₁, D₂, D₃, and D₄, and the precursor (7-dehydrositosterol) of vitamin D₅, are shown below:

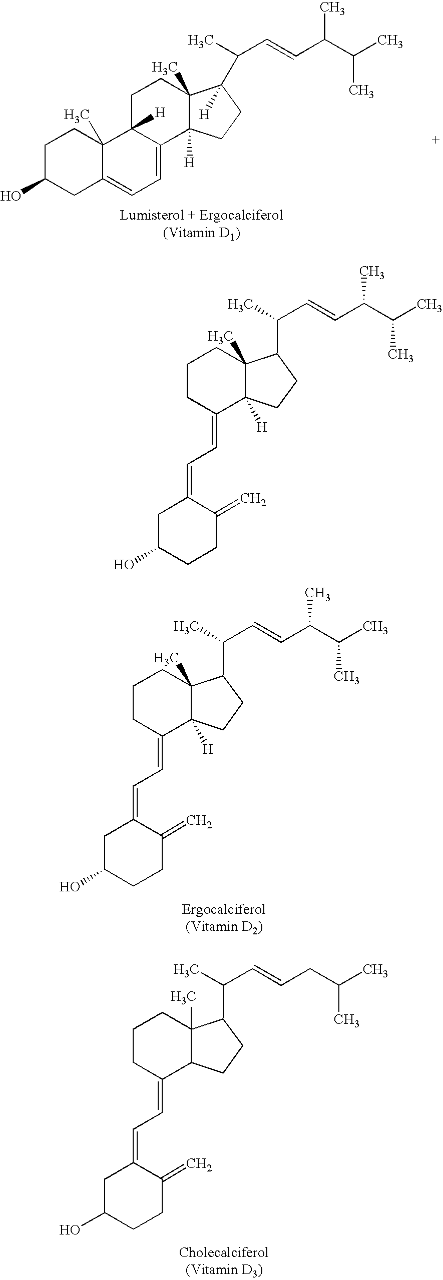

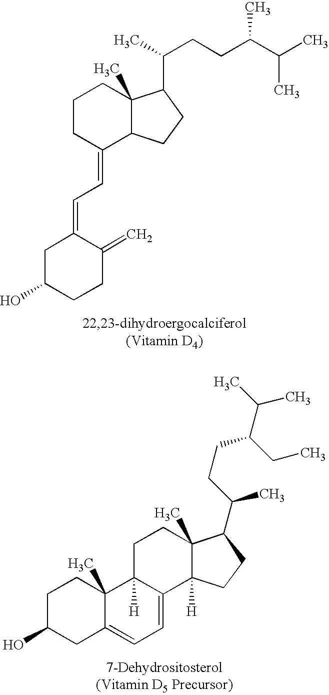

Without intending to be bound by any particular theory, it is believed that vitamin D has anti-osteoporotic, immunomodulatory, anti-carcinogenic, anti-psoriatic, anti-oxidant and mood-modulatory properties. Vitamin D may also regulate calcium homeostasis, is important in skeletal development and bone mineralization, helps maintain a healthy immune system, and contributes to regulation of cell growth and differentiation. Specifically, vitamin D₂ (i.e., calciferol) and vitamin D₃ (i.e., cholecalciferol), may facilitate increased absorption of calcium and phosphorus. Each of the aforementioned physiological processes may be improved through use of the composition.

The additional vitamin may also include the source of vitamin K. In one embodiment, the source of vitamin K is preferably present in the composition in an amount of approximately 22 IU per 100 grams of the composition and is commercially available from Spectrum Chemical Mfg Corp. of Gardena, Calif. However, the source of vitamin K may be present in any effective amount, as determined by one skilled in the art, depending on use of the composition and medical condition being treated.

Like vitamin D, there are many forms of vitamin K including, but not limited to, vitamin K₁, vitamin K₂, and vitamin K₃. In the instant invention, the source of vitamin K may include any and/or all of the forms of vitamin K. Most preferably, the source of vitamin K includes vitamin K₁, also known as phytonadione. For descriptive purposes only, the chemical structures of vitamin K₁, vitamin K₂, and vitamin K₃ are shown below:

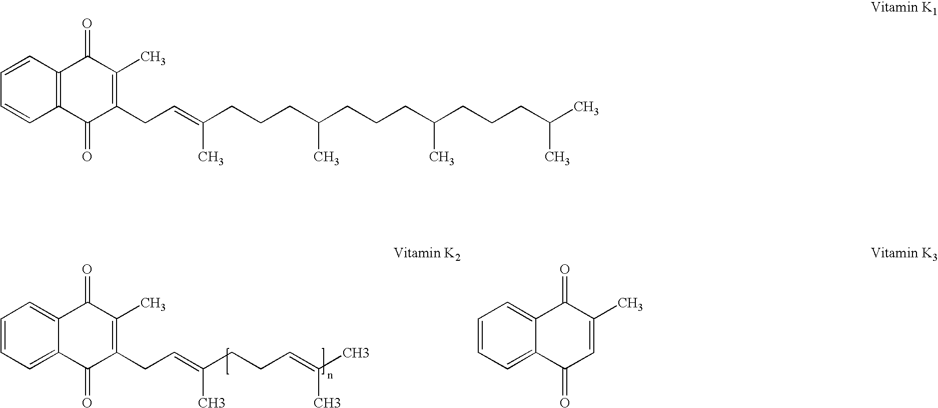

Vitamin K₂ may have isoprene units of varying lengths, identified by the subscript n. Preferably, n is an integer of from 4 to 13.

As is known in the art, vitamin K is a generic term that includes a group of compounds including a 2-methyl-1,4-naphthoquinone ring structure. Vitamin K is believed to have hemostatic activity and may have anti-osteoporotic, anti-oxidant and anti-carcinogenic properties, is a cofactor in many biochemical reactions, and contribute to kidney function, bone growth and repair, and blood clotting. Vitamin K may also prevent osteoporosis, may help treat cystic fibrosis, and may aid calcium entry into bone marrow. If the composition includes the source of the vitamin K, any or all of the aforementioned physiological processes may be improved.

In another embodiment of the invention, the additional vitamin may include a source of vitamin B. If so, the source of the vitamin B may include, but is not limited to, vitamin B₁ (e.g., thiamine), vitamin B₂ (e.g., riboflavin), vitamin B₃ (e.g., nicotinic acid and nicotinamide), vitamin B₅ (e.g., pantothenic acid), vitamin B₆ (e.g., pyridoxine and pyridoxamine), vitamin B₇ (e.g., vitamin H or biotin), vitamin B₉ (e.g., vitamin M, vitamin B-C, and folic acid), vitamin B₁₂, (e.g., cyanocobalamin), Vitamin B₁₃ (e.g., pyrimidinecarboxylic acid and orotic acid), vitamin B₁₄, vitamin B₁₅ (e.g., pangamic acid), vitamin B₁₆ (e.g., dimethylgycine), vitamin B₁₇ (e.g., amygdalin), vitamin B₁₈, vitamin B₁₉, vitamin B₂₀ (e.g., camitine), vitamin B₂₁, vitamin B₂₂, vitamin Bₕ, vitamin Bₘ, vitamin Bₚ, vitamin Bₜ, vitamin Bᵥ, vitamin B_{w}, vitamin Bₓ (e.g., p-aminobenzoic acid), lipoic acid, thiazolium salts of thiamin, thiamin hydrochloride and thiamin mononitrate, and combinations thereof. Preferably, the source of vitamin B includes a mixture of vitamin B₆, vitamin B₁₂, and vitamin B₉, which is commercially available from Archer Daniels Midland of Decatur, Ill. For descriptive purposes only, chemical structures of vitamin B₁, vitamin B₂, vitamin B₃, pantothenic acid, vitamin B₆, vitamin B₇, folic acid, and vitamin B₁₂ are shown below.

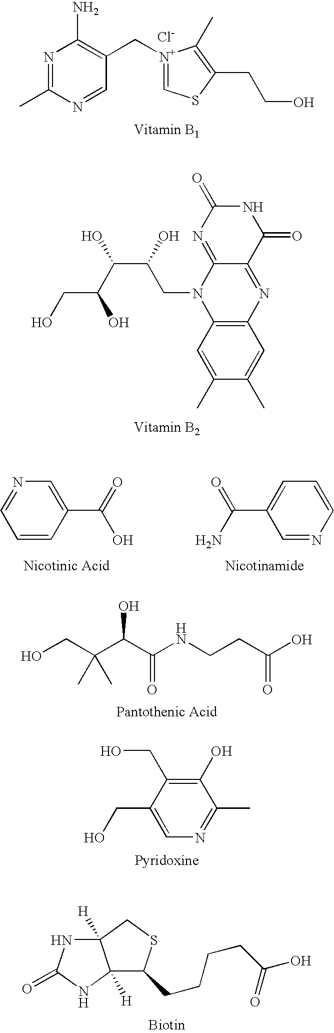

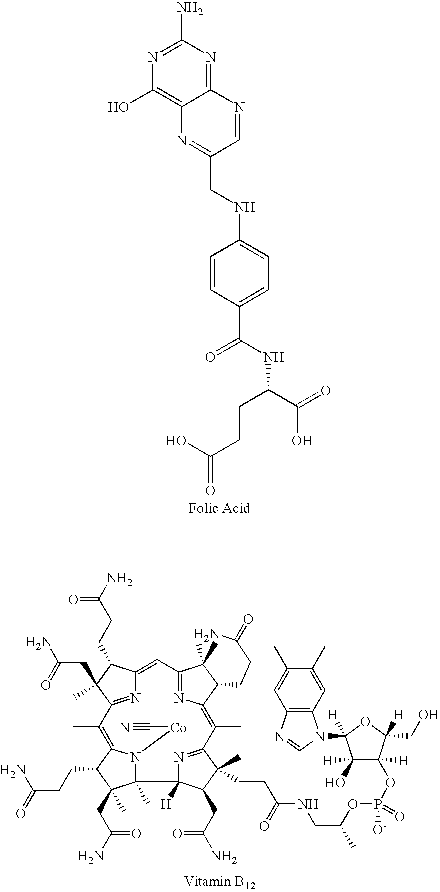

In one embodiment, the source of vitamin B is preferably present in the composition in an amount of from 0.02 to 0.06, more preferably of from 0.02 to 0.05, and most preferably of from 0.04 to 0.05, parts by weight per 100 parts by weight of the composition. However, the source of vitamin B may be present in any effective amount, as determined by one skilled in the art, depending on use of the composition and medical condition being treated.

Vitamin B₁ is water-soluble, includes a substituted pyrimidine ring bonded to a substituted thiazole ring via a methylene bridge, and may be known as aneurin and 3-[(4-amino-2-methyl-5-pyrimidinyl)methyl]-5-(2-hydroxyethyl)-4-methyl-thiazonium. Vitamin B₁ is also utilized in metabolism of carbohydrates, fat, and proteins, is used to produce ATP, may contribute to effective circulation, blood formation, and other metabolic processes, and may have anti-oxidant, erythropoietic, cognition and mood-modulatory, ergogenic, anti-atherosclerotic and detoxification properties. Further, it is contemplated that vitamin B₁ is essential as a co-factor in carbohydrate and amino acid metabolism, may protect against lipid peroxidation, and may protect against heavy metal toxicity.

Vitamin B₂, also known as riboflavin, riboflavine, 7,8-dimethyl-10-(11-D-ribityl)isoalloxazine, 7,8-dimethyl-10-(D-ribo-2,3,4,5-tetrahydroxypentyl)isoalloxazine and 7,8-dimethyl-10-ribitylisoalloxazine, is also utilized in metabolism and specifically amino acid and lipid metabolism. Vitamin B₂ may activate vitamin B₆ and folic acid and may alleviate symptoms associated with arthritis. Vitamin B₂ is also a component of many flavoenzymes which catalyze redox reactions which are essential to cellular respiration. Further, it is contemplated that vitamin B₂ has anti-oxidant and anti-atherosclerotic activity.

Vitamin B₃, also known as niacin, may include both nicotinic acid and nicotinamide. Nicotinic acid is also known as pyridine-3-carboxylic acid, 3-pyridinecarboxylic acid, pyridine-beta-carboxylic acid, antipellagra vitamin, and pellagra preventive factor. Nicotinamide is also known as pyridine-3-carboxamide, niacinamide and nicotinic acid amide. Both nicotinic acid and nicotinamide may contribute to energy production, lipid synthesis, signal transduction, and gene expression. Nicotinic acid may also have anti-hyperlipidemic and anti-atherogenic properties while nicotinamide may have anti-diabetogenic, anti-oxidant, anti-inflammatory and anti-carcinogenic properties and may exhibit activity against osteoarthritis and granuloma annulare.

Vitamin B₅ also known as pantothenic acid, D(+)-pantothenic acid, D-pantothenic acid, D(+)-N-(2,4-dihydroxy-3,3-dimethylbutyryl)-beta-alanine and (R)-N-(2,4-dihydroxy-3,3-dimethyl-1-oxobutyl)-beta-alanine, is required for energy production and is utilized in synthesis of acetylcholine. Vitamin B₅ may also contribute to both the synthesis and breakdown of fatty acids, phospholipids, and sphingolipids, to lowering of blood cholesterol, and to gene expression and signal transduction. Vitamin B₅ may further have anti-inflammatory, anti-arthritic, wound healing and anti-viral properties.

Vitamin B₆, as known in the art, may include a variety of compounds including, but not limited to, pyridoxine, pyridoxal, pyridoxamine, pyridoxine 5′-phosphate, pyridoxal 5′-phosphate, and pyridoxamine 5′-phosphate, and combinations thereof. Vitamin B₆ may contribute to peptide coupling, protein synthesis, in formation of serotonin, melatonin, and dopamine, in metabolism of amino acids and glycogen, and in synthesis of nucleic acids, hemoglobin, and sphingolipids. Additionally, it is contemplated that vitamin B₆ is involved as co-enzymes in synthesis of porphyrins and may be involved in energy production. Further, it is contemplated that vitamin B₆ has anti-neurotoxic, anti-atherogenic, immunomodulatory, anti-carcinogenic and mood-modulatory properties.

Referring now to vitamin B₇, also known as biotin, this vitamin may modulate glucose tolerance, to have anti-oxidant properties and to be involved in fatty acid synthesis, gluconeogenesis, energy production, metabolism of branched-chain amino acids, synthesis of purine nucleotides, and in DNA replication, transcription, and translation. Biotin, a bicyclic compound that includes a tetrahydrothiophene ring including sulfur and a valeric acid side chain and a second ring including a ureido group, has numerous stereoisomers. For purposes of the present invention, the vitamin B₇ may include any or all of the stereoisomers.

Vitamin B₉, also known as folic acid, is utilized in formation of red blood cells, in metabolism of proteins and lipids, and in the maintenance of nerves, skin, hair, and other tissue. It is contemplated that vitamin B₉ is also utilized in synthesis of DNA and RNA, in formation of L-methionine, in purine nucleotide synthesis, in interconversion of serine and glycine, in synthesis and utilization of formate, in metabolism of L-histidine to L-glutamic acid, in metabolism of dimethylglycine to sarcosine, and in metabolism of sarcosine to glycine. It is further contemplated that vitamin B₉ may have anti-atherogenic, anti-carcinogenic, neuroprotective and anti-depressant properties.

Vitamin B₁₂, for purposes of the present invention, may include a variety of cobalt compounds such as corrinoids including, but not limited to, cyanocobalamin, hydroxocobalamin, methylcobalamin, 5-deoxyadenosylcobalamin, and combinations thereof. Vitamin B₁₂ is believed to be critical to DNA replication and nervous system maintenance and may have anti-atherogenic, neuroprotective, anti-carcinogenic and detoxifying, anti-allergic, and mood-modulatory properties.

In addition to the mineral component and the vitamin component, the composition also includes the anti-inflammatory component including the source of lysine and the source of glutathione. Lysine, an essential amino acid, is utilized in production of carnitine, a nutrient responsible for converting fatty acids into energy for lowering cholesterol. Also, lysine may contribute to absorption and conservation of calcium, formation of collagen, repair of epithelial lesions and shingles, and may improve absorption and digestion of calcium thereby reducing bone loss associated with osteoporosis. Further, lysine may have analgesic properties. It is contemplated that through use of the composition including the lysine, each of the aforementioned physiological processes may be improved.

In the present invention, the source of lysine may include L and/or D enantiomers of lysine. Preferably, the source of lysine includes L-lysine which is commercially available from Spectrum Chemical Mfg Corp. of Gardena, Calif. For descriptive purposes only, a chemical structure of L-lysine is shown below:

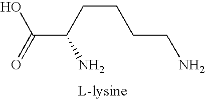

In one embodiment, the source of lysine is preferably present in the composition in an amount of from 0.2 to 0.4 and more preferably of from 0.2 to 0.3, parts by weight per 100 parts by weight of the composition. The source of lysine is commercially available from Spectrum Chemical Mfg Corp. of Gardena, Calif. The source of lysine may be present in any effective amount, as determined by one skilled in the art, depending on use of the composition and medical condition being treated.

Referring now to the source of glutathione, this source may include both L and/or D enantiomers of glutathione. Preferably, the source of glutathione includes L-glutathione. For descriptive purposes only, the chemical structure of glutathione is shown below:

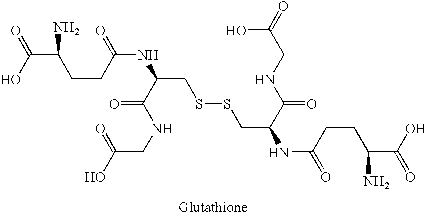

In one embodiment, the source of glutathione is preferably present in the composition in an amount of from 0.05 to 0.07 and more preferably of from 0.06 to 0.07, parts by weight per 100 parts by weight of the composition. The source of glutathione is commercially available from Spectrum Chemical Mfg Corp. of Gardena, Calif. The source of glutathione may be present in any effective amount, as determined by one skilled in the art, depending on use of the composition and medical condition being treated.

As is known in the art, glutathione is an anti-inflammatory agent, an anti-oxidant, and contributes to nutrient metabolism and regulation of gene expression, DNA and protein synthesis, cell growth, and immune responses. Glutathione is formed from a combination of three amino acids cysteine, glutamate, and glycine, and forms part of the natural antioxidant glutathione peroxidase, which is involved in DNA synthesis and repair, metabolism of toxins and carcinogens, enhancement of the immune system, and prevention of fat oxidation. Through use of the composition including the source of glutathione, each of the aforementioned physiological processes may be improved.

The anti-inflammatory component may also include a source of an additional anti-inflammatory selected from the group of methylsulfonyl methane (MSM), glucosamine, chondroitin, boswellia, devil's claw, yucca, ginkgo, and combinations thereof. It is contemplated that MSM is essential to joint health and function, contributes to skin nourishment and repair, and may retard skin aging. MSM may also facilitate diffusion of the composition and any or all of the components across the blood-brain barrier. The MSM may be present in any form and is commercially available from Spectrum Chemical Mfg Corp. of Gardena, Calif. In one embodiment, the source of the additional anti-inflammatory component including the MSM includes a 50% by volume solution and is preferably present in an amount of from 0.2 to 0.8, more preferably of from 0.2 to 0.6, and most preferably of from 0.4 to 0.6, parts by weight per 100 parts by weight of the composition. It is to be appreciated that the source of the additional anti-inflammatory component including the MSM may be present in any effective amount, as determined by one skilled in the art, depending on use of the composition and medical condition being treated. For descriptive purposes only, a chemical structure of MSM is shown below:

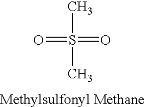

The source of the additional anti-inflammatory may also include glucosamine. Glucosamine, a natural compound that is found in healthy cartilage, is used to form collagen via formation of proteoglycans which are complex macromolecules including modified sugars such as glycosaminoglycans and/or mucopolysaccharides. The proteoglycans can be formed from hyaluronic acid (C₂₈H₄₂N₂Na₂O₂₃), of which glucosamine is an important precursor. Ingestion of glucosamine can ease symptoms of osteoarthritis and can inhibit bacteria growth and function. Each of the aforementioned physiological processes may be improved through use of the composition.

Preferably, in the composition, a source of glucosamine includes a salt. A non-limiting example of a suitable salt includes a sulfate (e.g., as glucosamine sulfate (C₆H₁₅NO₉S)). However, the source need not include a salt. In one embodiment, the source of the additional anti-inflammatory component including the glucosamine is preferably present in an amount of from 0.1 to 0.6 and more preferably of from 0.2 to 0.3, parts by weight per 100 parts by weight of the composition. The source of the additional anti-inflammatory component including the glucosamine may be present in any effective amount, as determined by one skilled in the art, depending on use of the composition and medical condition being treated. For descriptive purposes only, a chemical structure of glucosamine is shown below:

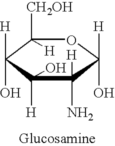

The source of the additional anti-inflammatory may also include chondroitin either with the glucosamine or independent of the glucosamine. Like the glucosamine, a source of chondroitin may include a salt such as a sulfate (e.g., chondroitin sulfate (C₁₃H₂₁NO₁₅S)). There are many forms of chondroitin sulfate that may be utilized in the present invention including, but not limited to, chondroitin sulfate A, B, C, D, and E, and combinations thereof. Chondroitin sulfate A includes linear repeating units including D-galactosamine (C₆H₁₃NO₅) and D-glucuronic acid (C₆H₁₀O₇) and is commonly known as chondroitin 4-sulfate. Chondroitin sulfate B includes linear repeating units of D-galactosamine and either L-iduronic acid (C₆H₁₀O₇) or D-glucuronic acid. Chondroitin sulfate C also includes linear repeating units of D-galactosamine and D-glucuronic acid. In chondroitin sulfate C, an amino group of D-galactosamine is acetylated to give N-acetyl-galactosamine (C₈H₁₅NO₆), and a sulfate group is esterified at the 6-position in N-acetyl-galactosamine. For descriptive purposes only, a generic chemical structure of a backbone of chondroitin is shown below wherein n is an integer. In some embodiments, R¹, R², and R³ may each independently include a hydrogen atom or an —SO₃H group.

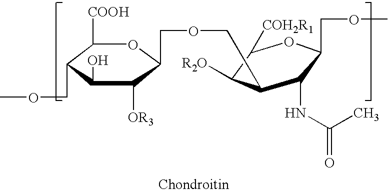

The source of the additional anti-inflammatory component including the chondroitin is preferably present in an amount of from 0.1 to 0.6 and more preferably of from 0.2 to 0.3, parts by weight per 100 parts by weight of the composition. However, the source of the additional anti-inflammatory component including the chondroitin may be present in any effective amount, as determined by one skilled in the art, depending on use of the composition and medical condition being treated.

Chondroitin, an abundant component in cartilage, contributes to resiliency, maintenance, structure, and function of cartilage, may contribute to pain relief of osteoarthritis, and may act as an anti-inflammatory. As such, it is contemplated that absorption and/or consumption of chondroitin can relieve symptoms of osteoarthritis.

The source of the additional anti-inflammatory may also include a source of boswellia extract. Boswellia extract may be derived from the Boswellia cartenii, Boswellia frereana, Boswellia bhau-dajaina, Boswellia serrata and Boswellia thurifera plant and may include boswellic acids. Without intending to be bound by any particular theory, it is believed that each of the boswellic acids includes a pentacyclic structure based on 12-ursen-24-oic acid. In one embodiment, the boswellic acids include, but are not limited to, a-hydroxyurs-12-ene-24-oic acid, 3a-acetoxyurs-12-ene-24-oic acid, 3a-hydroxyurs-12-ene-11-keto-24-oic acid, 3a-hydroxyurs-9,12-dien-24-oic acid, and combinations thereof. It is also believed that the boswellia extract may include 4-O-methyl-glucuronoarabinogalactan. The source of the boswellia extract is commercially available from San Francisco Herb & Natural Food Co. of Freemont, Calif.

As is known in the art, boswellia extract has anti-inflammatory, anti-atherosclerotic and anti-arthritic activities and is also believed to have sedative and analgesic properties. The source of the additional anti-inflammatory component including the boswellia extract is preferably present in an amount of from 0.02 to 0.04 and more preferably of from 0.03 to 0.04, parts by weight per 100 parts by weight of the composition. It is contemplated that the source of the additional anti-inflammatory component including the boswellia extract may be present in any effective amount, as determined by one skilled in the art, depending on use of the composition and medical condition being treated.

Additionally, the source of the additional anti-inflammatory may include Devil's Claw. Devil's claw is extracted from Harpogophytum procumbens, may have anti-inflammatory properties, may be used to relieve pain and stiffness of rheumatoid arthritis, and may relax muscles. Devil's claw may also be used for to treat skin lesions, and may be used to increase appetite and relieve indigestion. It is contemplated that through use of the instant composition, each of the aforementioned physiological processes may be improved.

Devil's Claw typically includes six primary glycosides as active ingredients. These glycosides include harpagoside, harpagide, procumbine, procumboside, 8-O-p-coumarylharpagide, and 6′-O-pcoumaroylprocumbide. For descriptive purposes only, chemical structures of harpagoside, harpagide, procumbine, procumboside, 8-O-p-coumarylharpagide, and 6′-O-p-coumaroylprocumbide are shown below.

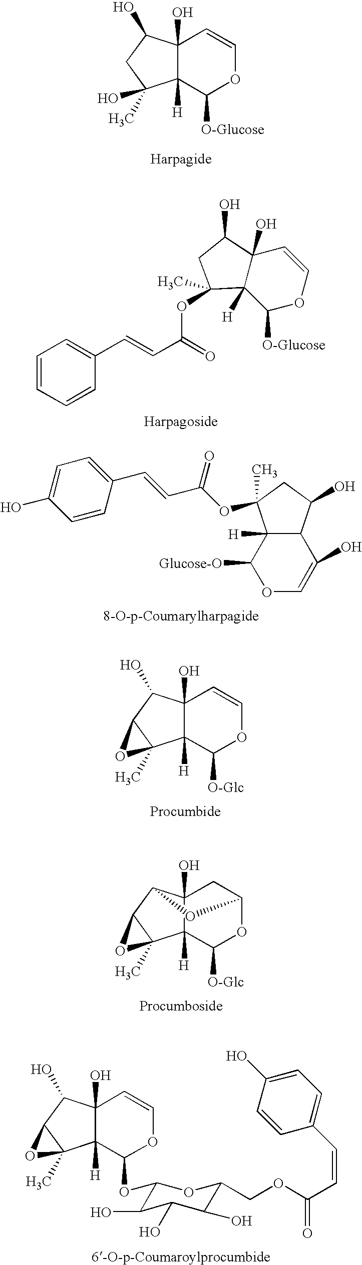

The source of the additional anti-inflammatory component including the Devil's claw is preferably present in an amount of from 0.1 to 0.2 and more preferably of from 0.1 to 0.12, parts by weight per 100 parts by weight of the composition. Alternatively, the source of the additional anti-inflammatory component including the Devil's Claw may be present in any effective amount, as determined by one skilled in the art, depending on use of the composition and medical condition being treated.

Further, the source of the additional anti-inflammatory may include yucca. Yucca may reduce cholesterol, ease symptoms of carpal tunnel syndrome, relieve arthritis, and relieve muscle soreness. A source of yucca may include yucca extract from any of the well known yucca plants such as Yucca schidigera. In one embodiment, the source of yucca includes vitamins A, B-complexes and vitamin C, and may further include copper, calcium, manganese, potassium, and fiber. Active ingredients in yucca typically include steroidal saponins as mono-desmosides having a single sugar component and as bi-desmosides having two sugar components. Generic chemical core structures of mono- and bi-desmosides commonly found in yucca are generically shown below:

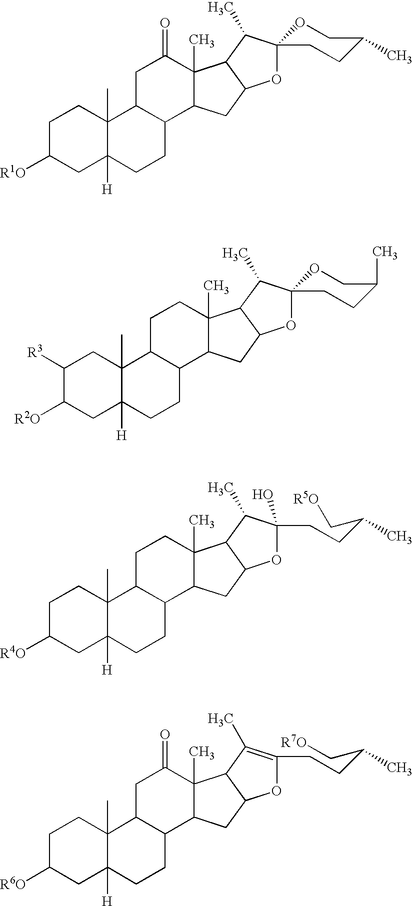

wherein R¹ can include glucose and xylose, R² can include glucose, xylose, and galactose, R³ can include hydrogen atoms or hydroxyl groups, R⁴ can include glucose, and xylose, R⁵ can include glucose, R⁶ can include glucose, xylose, and galactose, and R⁷ can include glucose.

Yucca may also include other active ingredients including, but not limited to, furostanol and spirostanol aglycones such as sasapogenin((25S)-5β-spirostan-3β-ol), markogenin((25S)-5β-spirostan-2β,3β-diol), smilagenin((25R)-5β-spirostan-3β-ol), samogenin((25R)-5β-spirostan-2β,3β-diol), gitogenin((25R)-5α-spirostan-2α,3β-diol) and neogitogenin((25S)-5α-spirostan-2α,3β-diol), spirostructures such as yuccaols A-E, trans-3,3′,5,5′-tetrahydroxy-4′-methoxystilbene, 3,4′,5-trihydroxystilbene, additional compounds such as spirobiflavonoids, and combinations thereof. For descriptive purposes only, chemical structures of trans-3,3′,5,5′-tetrahydroxy-4′-methoxystilbene and 3,4′,5-trihydroxystilbene are shown below.

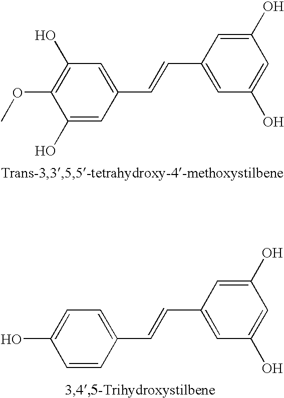

### Additionally, a generic chemical structure of a Yuccaol, representative of Yuccaols A-E, is also shown below.

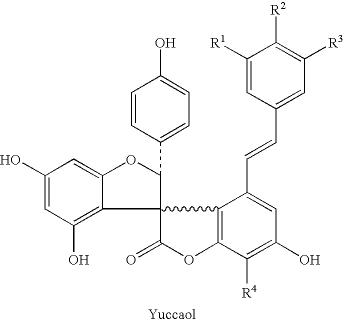

wherein R¹ may be a hydrogen atom or a hydroxyl group, R² may be a hydrogen atom, a hydroxyl group, or a methoxy group, R³ may be a hydrogen atom or a hydroxyl group, and R⁴ may be a hydrogen atom or a methoxy group.

The source of the additional anti-inflammatory component including the yucca is preferably present in an amount of 0.1 to 0.2 and more preferably of from 0.1 to 0.12, parts by weight per 100 parts by weight of the composition. However, the source of the additional anti-inflammatory component including the yucca may be present in any effective amount, as determined by one skilled in the art, depending on use of the composition and medical condition being treated.

The source of the additional anti-inflammatory may also include ginkgo, which is thought to thin blood, increase circulation and increase cognitive abilities. A source of ginkgo, which may include Ginkgo biloba, may also include many active ingredients including, but not limited to, flavonoids and terpene lactones, such as ginkgolides A, B, C, J, and M, bilobalide, quercetin, kaempferol, and combinations thereof. A generic chemical structure of ginkgolides is shown below wherein R¹ through R³ may each independently include a hydrogen atom or a hydroxy group. Generic chemical structures of bilobalide, quercetin, and kaempferol are also shown below.

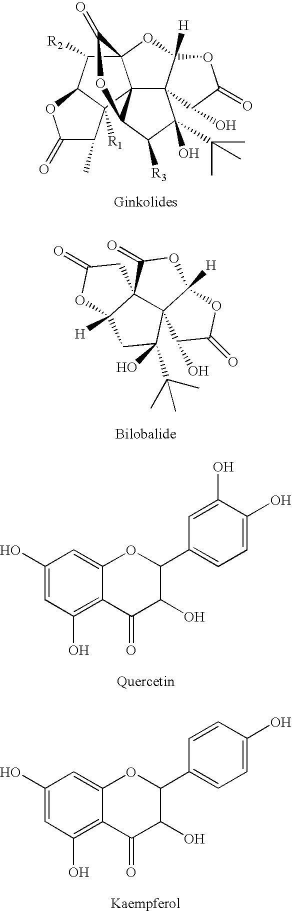

The source of the additional anti-inflammatory component including the ginkgo is preferably present in an amount of from 0.02 to 0.04 and more preferably of from 0.03 to 0.04, parts by weight per 100 parts by weight of the composition. It is to be appreciated that the source of the additional anti-inflammatory component including the ginkgo may be present in any effective amount, as determined by one skilled in the art, depending on use of the composition and medical condition being treated.

Referring now back to the anti-oxidant component of the composition, the anti-oxidant component includes a source of curcumin. Curcumin is an extract of turmeric root and is a known anti-oxidant, anti-inflammatory and anti-carcinogenic agent. Curcumin may slow development and growth of a number of types of cancer including prostate cancer. Curcumin may also scavenge reactive oxygen species such as superoxide anions and hydrogen peroxide, inhibit lipid peroxidation and inhibit oxidation of low-density lipoproteins. The source of curcumin in the present invention is not limited and may be in any form. One non-limiting source of curcumin includes Sabi White, commercially available from Sabinsa Corp. of Piscataway, N.J. For descriptive purposes only, a chemical structure of curcumin is shown below:

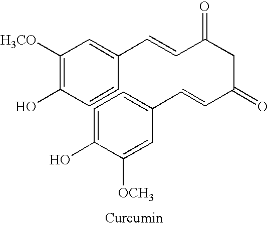

The source of curcumin is preferably present in the composition in an amount of from 0.1 to 0.6, more preferably of from 0.2 to 0.4, and most preferably of from 0.2 to 0.3, parts by weight per 100 parts by weight of the composition. Yet, it is to be appreciated that the source of the curcumin may be present in any effective amount, as determined by one skilled in the art, depending on use of the composition and medical condition being treated.

The anti-oxidant component may also include a source of an additional anti-oxidant selected from the group of butylated hydroxytoluene (BHT), a polyphenol, and combinations thereof. BHT may act as an anti-oxidant, to aid entry of the composition into blood, to reduce a potential of cancerous cell formation, and to help prevent breakdown of DNA, each of which may be improved by use of the instant invention. In one embodiment, the source of the additional anti-oxidant component including the BHT is included in the composition and is preferably present in an amount of from 0.1 to 0.6, more preferably of from 0.2 to 0.4, and most preferably of from 0.2 to 0.3, parts by weight per 100 parts by weight of the composition. The source of the additional anti-oxidant component including the BHT may be present in any effective amount, as determined by one skilled in the art, depending on use of the composition and medical condition being treated. For descriptive purposes only, a chemical structure of BHT is shown below:

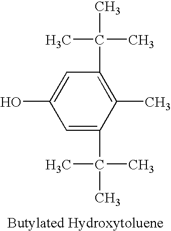

The source of the additional anti-oxidant may also include the polyphenol. A source of the polyphenol preferably includes a polyphenol of green tea and may include two or more polyphenols of green tea. Although there are many polyphenols in green tea that may be included in the composition, it is contemplated that the most biologically active polyphenols include (−)(−)epigallocatechin-3-gallate, (−)(−)epicatechin-3-gallate, (+)(−)catetchin, and (−)(−)catechin, whose chemical structures are shown below:

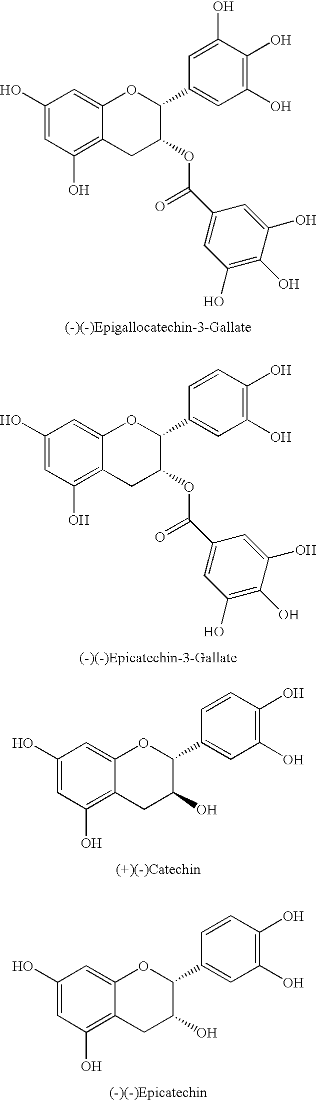

In one embodiment, the composition includes (−)(−)epigallocatechin-3-gallate. The source of the additional anti-oxidant component including the polyphenol is preferably present in an amount of from 0.01 to 0.1, more preferably of from 0.02 to 0.06, and most preferably of from 0.03 to 0.05, parts by weight per 100 parts by weight of the composition. It is to be appreciated that the source of the additional anti-oxidant component including the polyphenol may be present in any effective amount, as determined by one skilled in the art, depending on use of the composition and medical condition being treated.

Polyphenols of green tea may act as anti-oxidants, anti-bacterials, and as anti-mutagenic, anti-cancer, anti-atherogenic, and thermogenic agents. The polyphenols of green tea may also be useful to combat heart disease and stroke, in treatment of weight loss, in glucose homeostasis, and in treatment of epithelial infections. Specifically, the polyphenols of green tea may be useful to prevent sunburn and protect against DNA damage from UV light exposure. Polyphenols are believed to scavenge superoxide, hydroxyl and peroxyl radicals, inhibit lipid peroxidation, inhibit 2′-deoxyguanosine oxidation in DNA to 8-hydroxy-2′deoxyguanosine, and inhibit oxidation of low-density lipoproteins. Polyphenols may also induce apoptosis in tumor cell lines, inhibit angiogenesis, and are involved in the detoxification of xenobiotics, including chemical carcinogens. It is contemplated that each of the aforementioned physiological processes may be improved through use of the composition.

In addition to the mineral, vitamin, anti-inflammatory, and anti-oxidant components, the composition also includes an isoflavone component including a source of ipriflavone (7-isopropoxy isoflavone). As an isoflavone, ipriflavone is one of a family of phytoestrogens, which are estrogen mimics. Yet, it is believed that ipriflavone has no direct estrogenic activity and does not activate any estrogen receptors. Absorption and/or consumption of ipriflavone may improve general health, decrease risk for heart disease by helping to lower blood cholesterol levels, reduce incidence of hormone-dependent cancers, and retard bone loss due to osteoporosis. Specifically, ipriflavone can act as an anabolic agent, has been used in anti-osteoporosis drugs, and has been used to increase cardiovascular capacity.

Relative to an anabolic effect of ipriflavone, this effect may originate from an ability of ipriflavone to increase nitrogen retention, independent of calorie intake. Relative to increasing cardiovascular capacity, ipriflavone may induce calcium accumulation in mitochondria thereby positively influencing mitochondrial energetics and sparing cellular oxygen use. Relative to use in treating osteoporosis, ipriflavone is thought to inhibit bone-degrading osteoclast activity, a process also known as bone resorption, and also to stimulate bone building effects of estrogen and calcium on bone-forming (osteoblastic) cells. Ipriflavone may also stimulate osteoblast activity by down-regulation of endothelin receptors. For descriptive purposes only, a chemical structure of ipriflavone is shown below:

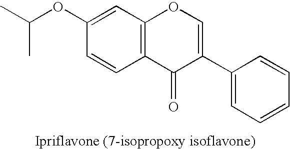

Preferably, the source of ipriflavone is present in the composition in an amount of from 0.1 to 1, more preferably of from 0.1 to 0.5, and most preferably of from 0.2 to 0.3, parts by weight per 100 parts by weight of the composition. One of skill in the art may include the source of the ipriflavone in any effective amount, depending on use of the composition and medical condition being treated.

It is contemplated that the isoflavone component may also include a source of additional isoflavones different from, and in addition to, the source of the ipriflavone. The source of the additional isoflavones may include, but is not limited to, genistein, daidzein, glycitein, and combinations thereof. As is known in the art, additional isoflavones such as genistein, daidzein, glycitein function differently from ipriflavone. For descriptive purposes only, chemical structures of genistein, daidzein, and glycitein are shown below:

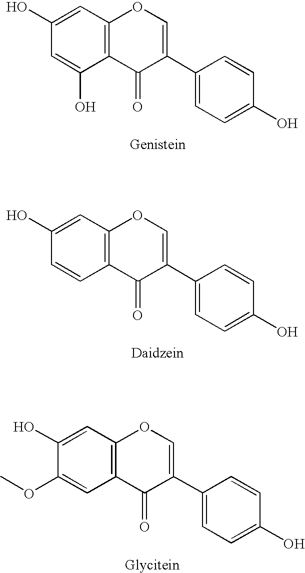

The composition may also include a dermatological additive selected from the group of sunflower oil, aloe vera, cocosin, tetrahydropiperine, and combinations thereof. Without intending to be bound by any particular theory, it is believed that the sunflower oil and the aloe vera act as skin soothing agents to promote skin healing and regeneration. It is also believed that cocosin and tetrahydropiperine promote absorption of the composition into skin. In one embodiment, the sunflower oil is preferably present in an amount of from 0.2 to 0.3 parts by weight per 100 parts by weight of the composition. The sunflower oil is commercially available from Archer Daniels Midland of Decatur, Ill. In another embodiment, the aloe vera is preferably present in an amount of from 1.2 to 1.4 parts by weight per 100 parts by weight of the composition. In a further embodiment, the tetrahydropiperine is present in an amount of from 0.002 to 0.004 parts by weight per 100 parts by weight of the composition. The tetrahydropiperin is commercially available from Sabinsa Corp. of Piscataway, N.J. under the trade name of Cosmoperine®. In still another embodiment, the cocosin is present in an amount of from 0.1 to 0.12 parts by weight per 100 parts by weight of the composition. The cocosin is commercially available from Sabinsa Corp. of Piscataway, N.J. It is to be appreciated that the dermatological additive may be present in any amount as determined by one of skill in the art and depending on the desired use of the composition.

The composition may also include a processing additive selected from the group of lethicin, gum, cellulose, talc, cornstarch, and combinations thereof. The processing additive may be included in the composition in any amount, as determined by one skilled in the art, depending on use of the composition and medical condition being treated. In one embodiment, the processing additive includes lethicin which preferably functions as an emulsifier and is preferably present in an amount of from 0.1 to 0.6 and more preferably of from 0.2 to 0.4, parts by weight per 100 parts by weight of the composition. The lethicin is commercially available from Cargill, Inc. of Minneapolis, Minn., Archer Daniels Midland of Decatur, Ill., and Central Soya Company, Inc. of Fort Wayne, Ind.

The processing additive may alternatively include the gum which preferably functions as a thickening agent is preferably present in an amount of from 0.01 to 0.06 and more preferably of from 0.04 to 0.06, parts by weight per 100 parts by weight of the composition. The gum is commercially available from Cargill, Inc. of Minneapolis, Minn. under the trade name of Versa gum. Without intending to be bound by any particular theory, it is believed that the gum interacts with water in the composition to at least partially gel the composition and develop a consistent and maintainable viscosity when the composition is applied.

In a further embodiment, the processing additive includes the cellulose. The cellulose is preferably present in an amount of from 0.01 to 0.04 and more preferably of from 0.01 to 0.02, parts by weight per 100 parts by weight of the composition. It is contemplated that the processing additive may include a combination of the gum and the cellulose wherein the combination is preferably present in an amount of from 0.04 to 0.06 parts by weight per 100 parts by weight of the composition.

If the processing additive includes the talc and/or cornstarch, the talc and/or cornstarch preferably homogenize the composition into a powder. The talc includes magnesium silicate and is commercially available from various sources. The cornstarch is commercially available from Archer Daniels Midland of Decatur, Ill. Although inclusion of the talc and/or cornstarch preferably homogenizes the composition into a powder, the composition is not limited to a powder. The composition may be an oil, a liquid, an ointment, a lotion, a paste, or a cream, and may still include the talc and/or the cornstarch. In one embodiment, the composition is water soluble. In another embodiment, the composition is a water and oil emulsion. The composition may have any color.

However, if the composition is a powder, the powder preferably includes granules. The granules may have any diameter, but preferably have a diameter of less than 180 μm and more preferably of less than 125 μm. If the composition is used with animals such as dogs, cats, and horses, the composition preferably includes the talc in place of the cornstarch and preferably includes at least 1 percent by weight of zinc oxide and less than 95 percent by weight of the talc. Alternatively, if the composition is used with humans, the composition preferably includes the cornstarch in place of the talc and includes at least 1 percent by weight of zinc oxide and less than 95 percent by weight of the cornstarch. It is contemplated that mixtures of talc and cornstarch may be utilized in the composition and that the amounts of the talc and/or cornstarch may be varied by one skilled in the art depending on desired application of the composition.

In addition to possibly including the processing additive, the composition may also include a fragrance selected from the group of frankincense, lavender, rosemary oil, and combinations thereof. In one embodiment, the frankincense is preferably present in an amount of from 0.1 to 0.4, more preferably of from 0.1 to 0.3, and most preferably of from 0.2 to 0.3, parts by weight per 100 parts by weight of the composition. In another embodiment, the fragrance includes the rosemary oil which is preferably present in an amount of from 0.1 to 0.4, more preferably of from 0.1 to 0.3, and most preferably of from 0.2 to 0.3, parts by weight per 100 parts by weight of the composition. It is to be appreciated that the fragrance may be included in the composition in any amount, as determined by one skilled in the art, depending on use of the composition and medical condition being treated.

Further, the composition may include a source of an anti-bacterial including a peroxide. Preferably, the peroxide includes hydrogen peroxide (H₂O₂). However, any peroxide or any anti-bacterial may be utilized in the present invention. Preferably, the hydrogen peroxide is present in an amount of from 0.001 to 0.003 parts by weight per 100 parts by weight of the composition. The source of the anti-bacterial may be included in the composition in any amount, as determined by one skilled in the art, depending on use of the composition and medical condition being treated.

Still further, the composition may include one or more solvents. The solvents may include acetone, isopropyl alcohol, maleic acid, and combinations thereof. The solvents may be present in the composition in any amount but are preferably minimized. In one embodiment, the acetone acts synergistically with the isopropyl alcohol to increase solubility of the curcumin in formation of the composition.

In all embodiments of the present invention, the composition may have any pH depending on the medical condition being treated. However, if the composition is used to treat fungal epithelial conditions, the pH is preferably at least 8, more preferably at least 9 and in one embodiment is at least 10. The pH of the composition may be tested by any method known in the art. If the composition is a powder, the pH of the powder can be determined by inserting litmus paper into the powder and determining the pH based on the color change of the litmus paper.

In addition to the composition, the present invention also provides a method of forming the composition. The method includes combining the mineral component, the vitamin component, the anti-inflammatory component, the anti-oxidant component, and the isoflavone component. In one embodiment, the composition is the powder and includes the granules and the method includes the step of reducing the diameter of the granules to less than 180 μm. In another embodiment, the method includes the step of reducing the diameter of the granules to less than 125 μm.

The method may also include the step of adjusting a pH of the composition to at least 9. In one embodiment, the method may include the step of adjusting the pH of the composition to at least 10. It is contemplated that the method alternatively may include adjusting the pH of the composition to any pH from 0 to 14 depending on the medical condition being treated. If the pH is adjusted positively, the pH is preferably adjusted via combining an alkaline metal hydroxide and/or an alkaline earth metal hydroxide in the composition. Preferably, the alkaline metal hydroxide or/or alkaline earth metal hydroxide is dissolved in water before adding to the composition such that uniform appearance of the composition is maintained. However, it is contemplated that the pH may be positively adjusted using compounds including, but not limited to, water, amine, hydrocarbons, and combinations thereof. If the pH is adjusted negatively, the pH is preferably adjusted via combining an acid in the composition. The acid may include, but is not limited to, the ascorbic acid, any strong acid known in the art (e.g., nitric acid, hydrochloric acid, hydrobromic acid, hydriodic acid, sulfuric acid, and perchloric acid) and combinations thereof. However, it is contemplated that the pH may be negatively adjusted using any compound known in the art.

In another embodiment of the method, the step of combining is further defined as combining the mineral component, the vitamin component, and the anti-oxidant component prior to introduction of the anti-inflammatory component and the isoflavone component. In this embodiment, the mineral component, the vitamin component, and the anti-oxidant component are combined prior to introduction of the anti-inflammatory component and the isoflavone component because it is contemplated that the sources of the lysine, glutathione, and ipriflavone may be air and temperature sensitive. As such, these sources are preferably introduced towards an end of the method.

In yet another embodiment, the step of combining is further defined as combining the source of ascorbic acid and the source of potassium to form a mixture prior to introduction of the source of calcium. In this embodiment, the method preferably further includes the step of adjusting a pH of the mixture to at least 7, more preferably to at least 7.4, even more preferably to at least 8, still more preferably to at least 9, and most preferably to at least 10. It is contemplated that combination of the source of ascorbic acid and the source of potassium prior to introduction of the source of calcium facilitates easier and more accurate adjustment of pH. In one embodiment, the alkaline metal hydroxide and/or alkaline earth metal hydroxide is combined with the source of ascorbic acid and the source of potassium prior to introduction of the source of calcium.

In still another embodiment, the method includes the step of combining the processing additive with the mineral component, the vitamin component, the anti-inflammatory component, the anti-oxidant component, and the isoflavone component. The method may also include the step of combining the fragrance with the mineral component, the vitamin component, the anti-inflammatory component, the anti-oxidant component, and the isoflavone component. The method may still further include the step of combining the processing additive with the fragrance and the mineral component, the vitamin component, the anti-inflammatory component, the anti-oxidant component, and the isoflavone component. It is also to be appreciated that the method may include the step of combining either or both of the dermatological additive and the solvent with any or all of the mineral component, the vitamin component, the anti-inflammatory component, the anti-oxidant component, the isoflavone component, the processing additive, the fragrance, and the additional minerals, vitamins, anti-oxidants, and anti-inflammatories.

### EXAMPLES

Two topical compositions (Compositions 1 and 2) of the present invention are formed according to the present invention. To form the Composition 1, 5.5 gallons of water are added to a first vessel in addition to 200 g of a Source of Butylated Hydroxy Toluene dissolved in water, 400 cc of a Source of Glucosamine, 400 cc of a Source of Chondroitin, 40 g of a Source of Polyphenol, 800 cc of a Source of Methylsulfonyl Methane, 200 cc of a 50% by weight solution of a Source of Devil's Claw in water, 200 cc of a 50% by weight solution of a Source of Yucca in water, 100 g of Lethicin, 2.72 g of Tetrahydropiperine, 100 g of Cocosin, 60 g of 3% Hydrogen Peroxide, 40 g of a Source of Vitamin B. 200 cc of a Source of Selenium, 200 cc of Rosemary Oil, 1200 cc of Aloe Vera, 200 cc of Frankincense, 200 cc of Sunflower Oil, 40 g of a Source of Potassium, and 40 g of Gum. After this addition, an additional amount of the water is added to the first vessel to increase the volume of the first mixture to 10 gallons. The first mixture is then heated to approximately 160° F.-165° F. for approximately 3 hours to maximize solubility of the Sources of the Butylated Hydroxy Toluene, Glucosamine, Chondroitin, Potassium, Polyphenol, Methylsulfonyl Methane, Devil's Claw, Yucca, Vitamin B, and Selenium, and the Lethicin, Tetrahydropiperine, Cocosin, Hydrogen Peroxide, Rosemary Oil, Aloe Vera, Frankincense, Sunflower Oil, and Gum, in the water. After heating, the first mixture is strained to remove any insoluble components, thereby providing a first filtrate.

Following, approximately 60 grams of Rare Earth are stirred into the first filtrate to maximize separation of any residual insoluble components from the first filtrate and absorb any off-color of the first filtrate. Once the Rare Earth settles to the bottom of the first vessel, the Rare Earth is filtered from the first filtrate and the first filtrate is stored in preparation for forming the composition.

In a second vessel, a second mixture is formed by combining one quart of water with 350 g of a Source of Calcium, 30 g of a Source of Ginkgo, 5000 IU of a Source of Vitamin E, 100,000 IU of a Source of Vitamin D, 20,000 IU of a Source of Vitamin K, 5 of Zinc Gluconate, as a portion of a Source of Zinc, approximately 1 g of a Source of Boron, 20 g of a Source of Manganese, 200 g of a Source of Ipriflavone, 30 g of a Source of Boswellia, 21 g of a Source of Copper including 20 g of Copper Gluconate and 1 gram of Copper Sulfate, 100 grams of Lethicin, and 20 g of Magnesium Sulfate, as a portion of a Source of Magnesium. Then, an additional amount of water is added to the second vessel to increase a volume of the second mixture to approximately 2.5 quarts. After the additional water is added, the second mixture is mixed and heated in a double boiler to approximately 160° F. to maximize solubility of the Sources of the Calcium, Ginkgo, Vitamin E, Vitamin D, Vitamin K, Boron, Manganese, Ipriflavone, Boswellia, and Copper, and the Zinc Gluconate, Magnesium Sulfate, and the Lethicin, in the water. After heating, the second mixture is strained through a cloth to remove any insoluble components, thereby providing a second filtrate. Subsequently, Rare Earth is added to the second filtrate to maximize separation of the insoluble components from the second filtrate. Once the Rare Earth settles to the bottom of the second vessel, the Rare Earth is filtered from the second filtrate. Following, the second filtrate is allowed to rest to precipitate any additional insoluble components. After precipitation, any insoluble components are separated from the second filtrate by pouring the second filtrate into a storage vessel where the second filtrate is stored in preparation for forming the composition.

In a third vessel, a third mixture is formed by combining approximately 2 quarts of water with 200 g of a Source of Curcumin, 200 cc of Acetone, and 200 cc of Isopropyl Alcohol at 160° F. The third mixture is stirred until well mixed and sediment is allowed to settle. A third filtrate is separated and is stored in preparation for forming the composition.

In a fourth vessel, a fourth mixture is formed by combining approximately 500 g of water with 200 g of a Source of Ascorbic Acid and approximately 80 g of a Source of Potassium. The fourth mixture is heated to approximately 160° F. The Source of Potassium is added in an amount such that a pH of the fourth mixture is raised to approximately 10. Once the pH of the fourth mixture is approximately 10, the fourth mixture is stored in preparation for forming the composition.

A fifth mixture is also formed in a fifth vessel by combining the first filtrate, the second mixture, the third filtrate, and the fourth mixture. Specifically, the fifth step includes adding 2 quarts of the second mixture, ½ gallon of water, 3 quarts of the first filtrate, the entire amount of the third filtrate, and the entire amount of the fourth mixture, to a stainless steel container, to form a solution which is then mixed. Following mixing, 80 cc of Isopropyl Alcohol, 12 g of Cellulose, 200 g of a Source of Lysine, 50 g of a Source of L-Glutathione, 50 g of Chelated Magnesium, as a portion of the Source of Magnesium, and 15 g of Maleic Acid, are added to the solution. Then, the solution is mixed at speeds from 2200 rpm to 7600 rpm and, if needed, an additional amount of the Source of the Potassium is added to the solution to raise a pH of the solution from approximately 7.4 to approximately 10. Additionally, approximately 10 grams of Zinc Oxide, as a portion of the Source of Zinc, is added to the solution.

To form the Composition 1, two quarts of the solution are added to a mixer with approximately 20 pounds of Talc to form a powder. To form the Composition 2, the same steps are followed as to form the Composition 1 except that the Talc is replaced with Cornstarch for human use. For both Compositions 1 and 2, the powder is mixed, ground, and sifted through a filter. The filter has mesh of a size between 80 and 120 to restrict a size of particles of the powder to less than approximately 180 μm. Amounts of each of the ingredients of the completed Compositions 1 and 2 are set forth in Table 1 below. The amounts are in grams unless otherwise indicated.

TABLE 1

| Component | | | Composition 1 | Composition 2 |
|---|---|---|---|---|
| Mineral | Source of | Calcium Lactate | 0.39 | 0.39 |
| | Calcium | | | |
| | Source of | Potassium | 0.16 | 0.16 |
| | Potassium | Hydroxide | | |
| Additional | Source of | Zinc Gluconate | 0.006 | 0.006 |
| Mineral | Zinc | | | |
| | | Zinc Oxide | 0.10 | 0.10 |
| | Source of | Elemental Boron | <0.001 | <0.001 |
| | Boron | | | |
| | Source of | Chelated | 0.022 | 0.022 |
| | Manganese | Manganese | | |
| | Source of | Magnesium | 0.03 | 0.03 |
| | Magnesium | Sulfate | | |
| | | Chelated | 0.055 | 0.055 |
| | | Magnesium | | |
| | Source of | Copper Gluconate | 0.022 | 0.022 |
| | Copper | | | |
| | | Copper Sulfate | 0.001 | 0.001 |
| | Source of | Elemental | 0.11 | 0.11 |
| | Selenium | Selenium | | |
| Vitamin | Source of | L-Ascorbic Acid | 0.22 | 0.22 |
| | Ascorbic Acid | | | |
| Additional | Source of | Cholated | 5.6 IU | 5.6 IU |
| Vitamin | Vitamin E | Vitamin E | | |
| | Source of | Vitamin D | 111 IU | 111 IU |
| | Vitamin D | | | |
| | Source of | Phytonadione | 22 IU | 22 IU |
| | Vitamin K | | | |
| | Source of | Vitamins | 0.044 | 0.044 |
| | Vitamin B | B₆, B₉, B₁₂ | | |
| Anti-Oxidant | Source of | Curcumin Extract | 0.22 | 0.22 |
| | Curcumin | | | |
| Additional | Source of | Butylated | 0.22 | 0.22 |
| Anti-Oxidant | Butylated | Hydroxy Toluene | | |
| | Hydroxy Toluene | | | |
| | Source of | Green Tea | 0.044 | 0.044 |
| | Polyphenol | | | |
| Anti- | Source of | L-Lysine | 0.22 | 0.22 |
| Inflammatory | Lysine | | | |
| | Source of | L-Glutathione | 0.06 | 0.06 |
| | Glutathione | | | |
| Additional | Source of | Methylsulfonyl | 0.5 | 0.5 |
| Anti- | Methylsulfonyl | Methane | | |
| Inflammatory | Methane | | | |
| | Source of | Glucosamine | 0.22 | 0.22 |
| | Glucosamine | Sulfate | | |
| | Source of | Chondroitin | 0.22 | 0.22 |
| | Chondroitin | Sulfate | | |
| | Source of | Devil's Claw | 0.11 | 0.11 |
| | Devil's Claw | Extract | | |
| | Source of | Boswellia Extract | 0.033 | 0.033 |
| | Boswellia | | | |
| | Source of | Yucca Extract | 0.11 | 0.11 |
| | Yucca | | | |
| | Source of | Ginkgo Biloba | 0.033 | 0.033 |
| | Ginkgo | | | |
| Isoflavone | Source of | Ipriflavone | 0.22 | 0.22 |
| | Ipriflavone | | | |
| Fragrance | Frankincense | | 0.22 | 0.22 |
| | Rosemary | | 0.22 | 0.22 |
| Anti-Bacterial | Hydrogen | | 0.002 | 0.002 |
| | Peroxide | | | |
| Processing | Lethicin | | 0.3 | 0.3 |
| Additive | | | | |
| | Gum | | 0.044 | 0.044 |
| | Cellulose | | 0.013 | 0.013 |
| | Cornstarch | | — | Balance |
| | Talc | | Balance | — |
| Dermatological | Sunflower Oil | | 0.22 | 0.22 |
| Additive | | | | |
| | Aloe Vera | | 1.33 | 1.33 |
| | Tetrahydropiperine | | 0.003 | 0.003 |
| | Cocosin | | 0.111 | 0.111 |
| Solvents | Acetone | | 0.22 | 0.22 |
| | Isopropyl Alcohol | | 0.31 cc | 0.31 cc |
| | Maleic Acid | | 0.017 | 0.017 |

The Source of Calcium includes Calcium Lactate and is commercially available from Jungbauer Inc. of Newton Centre, Mass., under the trade names of Purecal T, Purecal O. and Purecal U and also commercially available from Glambia Foods Inc., of Monroe, Wis.

The Source of Potassium includes Potassium Hydroxide which is commercially available from Spectrum Chemical Mfg Corp. of Gardena, Calif.

The Source of Zinc includes Zinc Gluconate and Zinc Oxide, each of which is commercially available from Spectrum Chemical Mfg Corp. of Gardena, Calif.

The Source of Boron includes Elemental Boron (powder) and is commercially available from Spectrum Chemical Mfg Corp. of Gardena, Calif.

The Source of Manganese includes Chelated Manganese. The Chelated Manganese is magnesium chelated with ethylenediamine tetraacetic acid and is commercially available from Spectrum Chemical Mfg Corp. of Gardena, Calif.

The Source of Magnesium includes Magnesium Sulfate. The Source of Magnesium also includes Chelated Magnesium which is Dihydrogen Magnesium Ethylenediaminetetraacetate. Both the Magnesium Sulfate and the Chelated Magnesium are commercially available from Spectrum Chemical Mfg Corp. of Gardena, Calif.

The Source of Copper includes Copper Gluconate and Copper Sulfate each of which is commercially available from Spectrum Chemical Mfg Corp. of Gardena, Calif.

The Source of Selenium includes Elemental Selenium and is commercially available from Spectrum Chemical Mfg Corp. of Gardena, Calif.

The Source of Ascorbic Acid includes L-Ascorbic Acid and is commercially available from Spectrum Chemical Mfg Corp. of Gardena, Calif.

The Source of Vitamin E includes Cholated Vitamin E and is commercially available from Spectrum Chemical Mfg Corp. of Gardena, Calif.

The Source of Vitamin D is Vitamin D itself and is commercially available from Spectrum Chemical Mfg Corp. of Gardena, Calif.

The Source of Vitamin K includes Phytonadione and is commercially available from Spectrum Chemical Mfg Corp. of Gardena, Calif.

The Source of Vitamin B includes Vitamin B₆, Vitamin B₉ (Folic Acid), and Vitamin B₁₂, all of which are commercially available from Archer Daniels Midland of Decatur, Ill.

The Source of Curcumin includes Curcumin Extract and is commercially available from Sabinsa Corp. of Piscataway, N.J. under the trade name Sabi White.

The Source of Butylated Hydroxy Toluene is Butylated Hydroxy Toluene itself and is commercially available from Spectrum Chemical Mfg Corp. of Gardena, Calif.

The Source of Polyphenol includes Green Tea and is commercially available from Templar Food Products of New Providence, N.J.

The Source of Lysine includes L-Lysine and is commercially available from Spectrum Chemical Mfg Corp. of Gardena, Calif.

The Source of Glutathione includes L-Glutathione and is commercially available from Spectrum Chemical Mfg Corp. of Gardena, Calif.

The Source of Methylsulfonyl Methane is Methylsulfonyl Methane itself and is commercially available from Spectrum Chemical Mfg Corp. of Gardena, Calif.

The Source of Glucosamine includes Glucosamine Sulfate and is commercially available from Spectrum Chemical Mfg Corp. of Gardena, Calif.

The Source of Chondroitin includes Chondroitin Sulfate and is commercially available from Spectrum Chemical Mfg Corp. of Gardena, Calif.

The Source of Devil's Claw includes Devil's Claw Extract and is commercially available from Spectrum Chemical Mfg Corp. of Gardena, Calif.

The Source of Boswellia includes Boswellia Extract and is commercially available from Spectrum Chemical Mfg Corp. of Gardena, Calif.

The Source of Yucca includes Yucca Extract and is commercially available from Spectrum Chemical Mfg Corp. of Gardena, Calif.

The Source of Ginkgo includes Ginkgo Biloba and is commercially available from San Francisco Herb & Natural Food Co. of Freemont, Calif.

The Source of Ipriflavone is Ipriflavone itself and is commercially available from Sabinsa Corp. of Piscataway, N.J.

The Frankincense is commercially available from Aromaland Inc. of Sante Fe, N. Mex.

The Rosemary Oil is commercially available from Aromaland Inc. of Sante Fe, N. Mex.

The Lethicin is commercially available from Archer Daniels Midland of Decatur, Ill.

The Gum is commercially available from Cargill, Inc. of Minneapolis, Minn. under the trade name of Versa Gum.

The Cellulose is commercially available from various paint ingredient suppliers.

The Talc includes 100% magnesium silicate.

The Cornstarch is commercially available from Archer Daniels Midland of Decatur, Ill.

The Sunflower Oil is commercially available from Archer Daniels Midland of Decatur, Ill.

The Aloe Vera is commercially available from a wide variety of sources.

The Tetrahydropiperine is commercially available from Sabinsa Corp. of Piscataway, N.J. under the trade name Cosmoperine®.

The Cocosin is commercially available from Sabinsa Corp. of Piscataway, N.J.

The Acetone is commercially available from Spectrum Chemical Mfg Corp. of Gardena, Calif.

The Isopropyl Alcohol is commercially available from Spectrum Chemical Mfg Corp. of Gardena, Calif.

The Maleic Acid is commercially available from Spectrum Chemical Mfg Corp. of Gardena, Calif.

After formation of the completed Composition 1, a sample is applied to the skin of a horse manifesting a fungal epithelial infection, as determined by a licensed veterinarian. Specifically, the sample is applied to the fungal epithelial infection a single time. After the single application, the licensed veterinarian reports that the Composition 1 eliminates the fungal epithelial infection on contact.

Also, after formation of the completed Composition 2, a sample is applied to the skin of a human patient suffering from carpal tunnel syndrome, as determined by a licensed medical doctor (M.D.). The sample is repeatedly applied to the skin of the human patient approximately 20 times over five days. After repeated application, the human patient reports that Composition 2 decreases symptoms of the carpal tunnel syndrome.

Further, after formation of the completed Composition 2, a sample is applied to the skin of a human patient suffering from tendonitis, as determined by a licensed medical doctor (M.D.). The sample is repeatedly applied to the skin of the human patient approximately 20 times over five days. After repeated application, the human patient reports that Composition 2 decreases symptoms of the tendonitis.

Obviously, many modifications and variations of the present invention are possible in light of the above teachings. The invention may be practiced otherwise than as specifically described within the scope of the appended claims.

1. A topical composition for use in treating medical conditions, said composition comprising:
a mineral component comprising a source of calcium and a source of potassium;
a vitamin component comprising a source of ascorbic acid;
an anti-inflammatory component comprising a source of lysine and a source of glutathione;
an anti-oxidant component comprising a source of curcumin; and
ipriflavone.

2. A topical composition as set forth in claim 1 wherein said vitamin component further comprises a source of an additional vitamin selected from the group of vitamin E, vitamin D, vitamin K, vitamin B, and combinations thereof.

3. A topical composition as set forth in claim 1 wherein said mineral component further comprises a source of an additional mineral selected from the group of zinc, boron, manganese, magnesium, copper, selenium, and combinations thereof.

4. A topical composition as set forth in claim 3 wherein said mineral component further comprises magnesium sulfate.

5. A topical composition as set forth in claim 1 wherein said anti-oxidant component further comprises a source of an additional anti-oxidant selected from the group of butylated hydroxytoluene, a polyphenol, and combinations thereof.

6. A topical composition as set forth in claim 1 wherein said anti-inflammatory component further comprises a source of an additional anti-inflammatory selected from the group of methylsulfonyl methane, glucosamine, chondroitin, devil's claw, yucca, ginkgo, boswellia, and combinations thereof.

7. A topical composition as set forth in claim 1 further comprising a processing additive selected from the group of lethicin, gum, cellulose, talc, cornstarch, and combinations thereof.

8. A topical composition as set forth in claim 1 further comprising a dermatological additive selected from the group of sunflower oil, aloe vera, cocosin, tetrahydropiperine, and combinations thereof.

9. A topical composition as set forth in claim 1 further comprising a fragrance selected from the group of frankincense, rosemary, lavender, and combinations thereof.

10. A topical composition as set forth in claim 1 further comprising a source of an anti-bacterial comprising a peroxide.

11. A topical composition as set forth in claim 1 wherein said composition is a powder comprising granules.

12. A topical composition as set forth in claim 11 wherein said granules have a diameter of less than 180 μm.

13. A topical composition as set forth in claim 11 wherein said granules have a diameter of less than 125 μm.

14. A topical composition as set forth in claim 1 having a pH of at least 9.

15. A topical composition as set forth in claim 1 wherein said source of calcium is present in an amount of from 0.3 to 0.4 parts by weight per 100 parts by weight of said topical composition.

16. A topical composition as set forth in claim 15 wherein said source of potassium is present in an amount such that the pH of the composition is at least 7.4.

17. A topical composition as set forth in claim 16 wherein said source of ascorbic acid is present in an amount of from 0.2 to 0.3 parts by weight per 100 parts by weight of said topical composition.

18. A topical composition as set forth in claim 17 wherein said source of lysine is present in an amount of from 0.2 to 0.3 parts by weight per 100 parts by weight of said topical composition.

19. A topical composition as set forth in claim 18 wherein said source of glutathione is present in an amount of from 0.06 to 0.07 parts by weight per 100 parts by weight of said topical composition.

20. A topical composition as set forth in claim 19 wherein said source of curcumin is present in an amount of from 0.2 to 0.4 parts by weight per 100 parts by weight of said topical composition.

21. A topical composition as set forth in claim 20 wherein said ipriflavone is present in an amount of from 0.1 to 0.5 parts by weight per 100 parts by weight of said topical composition.

22. (canceled)

23. A method of forming a topical composition for use in treating medical conditions, said method comprising combining a mineral component comprising a source of calcium and a source of potassium, a vitamin component comprising a source of ascorbic acid, an anti-inflammatory component comprising a source of lysine and a source of glutathione, an anti-oxidant component comprising a source of curcumin, and an isoflavone component comprising a source of ipriflavone.

24. A method as set forth in claim 23 wherein the composition is a powder comprising granules.

25. A method as set forth in claim 23 further comprising the step of adjusting a pH of the composition to at least 9.

26. A method as set forth in claim 23 wherein the step of combining is further defined as combining the mineral component, the vitamin component, and the anti-oxidant component prior to introduction of the anti-inflammatory component and isoflavone component.

27. A method as set forth in claim 23 wherein the step of combining is further defined as combining the source of ascorbic acid and the source of potassium to form a mixture prior to introduction of the source of calcium.

28. A method as set forth in claim 27 further comprising the step of adjusting a pH of the mixture to at least 7.

29. A method as set forth in claim 23 wherein the vitamin component further comprises a source of an additional vitamin selected from the group of vitamin E, vitamin D, vitamin K, vitamin B, and combinations thereof.

30. A method as set forth in claim 23 wherein the mineral component further comprises a source of an additional mineral selected from the group of zinc, boron, manganese, magnesium, copper, selenium, and combinations thereof.

31. A method as set forth in claim 23 wherein the mineral component further comprises a source of magnesium sulfate.

32. A method as set forth in claim 23 wherein the anti-oxidant component further comprises a source of an additional anti-oxidant selected from the group of butylated hydroxytoluene, a polyphenol, and combinations thereof.

33. A method as set forth in claim 23 wherein the anti-inflammatory component further comprises a source of an additional anti-inflammatory selected from the group of methylsulfonyl methane, glucosamine, chondroitin, devil's claw, yucca, ginkgo, and combinations thereof.

34. A method as set forth in claim 23 further comprising the step of combining a processing additive selected from the group of lethicin, gum, cellulose, talc, cornstarch, and combinations thereof.

35. A method as set forth in claim 23 further comprising the step of combining a dermatological additive selected from the group of sunflower oil, aloe vera, cocosin, tetrahydropiperine, and combinations thereof.

36. A method as set forth in claim 23 further comprising the step of combining a fragrance selected from the group of frankincense, rosemary, lavender, and combinations thereof.

37. A topical composition as set forth in claim 1 having a pH of greater than 7.

38. A topical composition as set forth in claim 1 having a pH of from 7.4 to 10.
